(19) United States
(12) Patent Application Publication
(10) Pub. No.: US 2013/0247248 A1
(43) Pub. Date: Sep. 19, 2013

(54) TRANSCRIPTION FACTORS IN PLANTS RELATED TO LEVELS OF NITRATE AND METHODS OF USING THE SAME

(71) Applicant: PONTIFICIA UNIVERSIDAD CATÓLICA DE CHILE, SANTIAGO (CL)

(72) Inventors: Rodrigo Antonio Gutierrez Ilabaca, Santiago (CL); José Miguel Alvarez Herrera, Santiago (CL)

(73) Assignee: PONTIFICIA UNIVERSIDAD CATÓLICA DE CHILE, SANTIAGO (CL)

(21) Appl. No.: 13/785,105

(22) Filed: Mar. 5, 2013

Related U.S. Application Data
(60) Provisional application No. 61/606,852, filed on Mar. 5, 2012.

(51) Int. Cl. C07K 14/415; C12N 15/82

(52) U.S. Cl. CPC C07K 14/415; C12N 15/8261
USPC 800/290; 504/315; 800/298; 536/23.1; 435/419

(57) ABSTRACT

This disclosure concerns plant nitrogen responses. Embodiments concern regulatory factors that contribute to the response to nitrogen sources and/or their metabolites in plants.

### CROSS-REFERENCE TO RELATED APPLICATION

This application claims the benefit of U.S. Provisional Patent Application Ser. No. 61/606,852, filed Mar. 5, 2012, the disclosure of which is hereby incorporated herein in its entirety by this reference.

### FIELD OF THE DISCLOSURE

The present disclosure relate to the influence of nitrogen levels on gene expression in plants. Embodiments relate to genes, and regulatory factors encoded thereby, that contribute to the response of a plant to nitrogen-containing molecules in the environment.

### BACKGROUND

Nitrogen (N) is an essential macronutrient for plants, and its availability is a major limiting factor for plant growth and crop production. Nitrate (NO₃) is the main source of inorganic nitrogen for plants in aerobic soils. See, e.g., Crawford and Glass (1998) Trends Plant Sci. 3:389-95; Hirsch and Sussman (1999) Trends Biotechnol. 17:356-61. Nitrate is taken up by plant roots through specific transporters, such as AtNRT1.1 (Tsay et al. (1993) Cell 72:705-13); AtNRT1.2 (Huang et al. (1999) Plant Cell 11:1381-92); AtNRT2.1 (Little et al. (2005) Proc. Natl. Acad. Sci. USA 102:13693-8); and AtNRT2.2 (Li et al. (2007) Plant Physiol. 143:425-33). Once inside a root cell, nitrate can be reduced to nitrite (NO₂⁻), and then ammonium (NH₄⁺), by the action of nitrate reductase (NR) and nitrite reductase (NIR), respectively. Crawford and Glass (1998), supra. The resulting ammonium is then assimilated into glutamic acid and glutamine by the glutamine synthetase (GS) and glutamate synthase (GOGAT) cycle. Stitt (1999) Curr. Opin. Plant Biol. 2:178-86.

In Arabidopsis, nitrate serves as a nutrient, and also as a potent signal to control gene expression and developmental responses. Vidal and Gutierrez (2008) Curr. Opin. Plant Biol. 11:521-9; Krouk et al. (2010a) Curr. Opin. Plant Biol. 13:266-73; Tsay et al. (2011) Annu Rev. Plant Biol. 62:207-26. Nitrate, as well as nitrite, can act as signals to regulate global gene expression in Arabidopsis. Wang et al. (2007) Plant Physiol. 145:1735-45. Very little is known about the signaling effects of nitrite and no specific function has been associated with nitrite-responsive genes. However, it has been proposed that nitrate-sensing system in Arabidopsis roots recognizes nitrite as well as nitrate, because both signals have an extensive overlapping response. Id.

The nitrate-responsive genes in Arabidopsis are many and varied, including nitrate transporters, NR and NIR, transcription factors, stress response genes, carbon (C) assimilation enzymes involved in N/C balance, and genes whose products participate in signal transduction pathways. Vidal and Gutierrez (2008), supra; Krouk et al. (2010a), supra; Tsay et al. (2010), supra. Transcriptomics analyses have identified a number of Arabidopsis nitrate-responsive genes. Wang et al. (2003) Plant Physiol. 132:556-7; Scheible et al. (2004) Plant Physiol. 136:2483-99; Wang et al. (2004) Plant Physiol. 136:2512-22; Gutierrez et al. (2007) Genome Biol. 8:R7. However, only a handful of regulatory factors involved in evoking nitrate responses have been identified.

The nitrate transporter, NRT1.1, has been proposed as the nitrate sensor in Arabidopsis. Ho et al. (2009) Cell 138:1184-94. In addition, a NIN-like protein 7 transcription factor has been described as involved in the regulation of nitrate assimilation (Castaings et al. (2009) Plant J. 57:426-35), while the ANR1 MADS-box gene has been identified as a regulator of lateral root growth in response to external nitrate (Zhang and Forde (1998) Science 279:407-9). Furthermore, a calcineurin B-like (CBL)-interacting protein kinase (CIPK) gene, CIPK8, was found to be involved in nitrate sensing. Hu et al. (2009) Plant J. 57:264-78. Also, the LBD37/38/39 transcription factors that repress N-responsive genes were found to be required for nitrate uptake and assimilation. Rubin et al. (2009) Plant Cell 21:3567-84. More recently, the nitrate responsive miR393/AFB3 module was found to control root system architecture in response to external and internal N availability in Arabidopsis (Vidal et al. (2010b) Proc. Natl. Acad. Sci. USA 107:4477-82), and a cell-specific regulation by glutamine in roots that is responsible for the control of lateral root architecture was attributed to the miR167/ARF8 module (Gifford et al. (2008) Proc. Natl. Acad. Sci. USA 105:803-8).

### BRIEF SUMMARY OF THE DISCLOSURE

Described herein are novel nitrogen response regulatory factors, TGA1 and TGA4. TGA1 and TGA4 mediate nitrogen regulation of the expression of genes involved in nitrogen uptake and reduction. In embodiments, these transcription factors may be used to modify the absorption and assimilation of nitrogen, affect the growth of root tissue, and/or influence the growth and productivity of plants.

Primary and lateral root growth was observed to be affected in tga1/tga4 double mutants, demonstrating a positive role of promoting root growth in the presence of nitrate. Thus, in some embodiments, TGA1 and/or TGA4 may be utilized to promote primary and/or lateral root growth in a plant, for example, under nitrogen-limiting conditions.

Nearly all (97%) of the genes identified in tga1/tga4 double mutant plants as under the regulatory control of TGA1 and TGA4 are regulated by nitrogen. Thus, in some embodiments, TGA1 and/or TGA4 may be utilized to the expression of a gene (e.g., a gene identified in FIG. 5) in response to nitrogen. For example, in particular embodiments TGA1 and/or TGA4 may be utilized to affect the expression of the nitrate transporters, NRT2.1, NRT2.2, and/or the nitrite reductase, NIR. In particular embodiments, TGA1 and/or TGA4 may be utilized to the expression of a gene operably linked to a TGA1 or TGA4 binding motif.

In some embodiments, a TGA1 polypeptide used in a method described herein may comprise an amino acid sequence selected from the group consisting of SEQ ID NOs:1-10, or a homolog sharing sequence identity with one or more of SEQ ID NOs:1-10. In some embodiments, a TGA4 polypeptide used in a method described herein may comprise an amino acid sequence selected from the group consisting of SEQ ID NOs:11-14, or a homolog sharing sequence identity with one or more of SEQ ID NOs:11-14. In some embodiments, a nucleic acid used in a method described herein may comprise a nucleotide encoding a TGA1 or TGA4 polypeptide.

Some embodiments include a transgenic plant or a progeny thereof, comprising a heterologous TGA1 polypeptide and/or a TGA1-encoding nucleic acid, and/or a heterologous TGA4 polypeptide and/or a TGA4-encoding nucleic acid. In particular embodiments, a heterologous TGA1- and/or TGA4-encoding nucleic acid is expressed in the transgenic plant or progeny thereof. In certain embodiments, the tissue wherein the heterologous TGA1- and/or TGA4-encoding nucleic acid is expressed is root. Methods according to some embodiments include growing the foregoing transgenic plant or progeny thereof under environmental conditions with a limiting nitrogen source. In some examples, a transgenic plant or progeny comprising a heterologous TGA1 and/or TGA4 polypeptide and/or a heterologous TGA1- and/or TGA4-encoding nucleic acid exhibits increased growth and/or tolerance under nitrogen limiting conditions (e.g., low nitrogen conditions).

Some embodiments include methods for producing a plant in which the primary and/or lateral root growth is promoted. Such methods may comprise, for example and without limitation, introducing a TGA1- and/or TGA4-encoding nucleic acid (e.g., in a vector) into a plant, or a cell or tissue thereof; optionally culturing the cell or tissue to regenerate a plant; and selecting the plant for promoted growth of primary and/or lateral roots. In some examples, the selected plant is cultivated under nitrogen-limiting conditions.

In some embodiments, a plant used in a method described herein may be an Arabidopsis sp. In some embodiments, the plant may be selected from the group consisting of Brassicaceae; Fabaceae; Poaceae; Solanaceae; Vitaceae; Euphorbiaceae; Salicaceae; and Myrtaceae. Plants, plant materials, plant cells, and seeds obtained by any of the aforementioned methods are also a feature of particular embodiments.

### BRIEF DESCRIPTION OF THE FIGURES

FIG. 1 includes illustrations of the responses of TGA1 and TGA4 to nitrate, and to a signal downstream of nitrate reduction. FIG. 1A includes the nitrate response of TGA1 in the wild-type “Col-0” plant. FIG. 1B includes the nitrate response of TGA4 in the Col-0 plant. FIG. 1C includes the nitrate response of TGA1 in the NR-null mutant plant. FIG. 1D includes the nitrate response of TGA4 in the NR-null mutant plant. Nitrite response of TGA1 (E) and TGA4 (F) genes. Ammonium response of TGA1 (G) and TGA4 (H) genes. The asterisk (*) indicates means that significantly differ between control and treatment conditions (P<0.05).

FIG. 2 includes illustrations of the nitrite responses of TGA1 and TGA4, as compared to ammonium. FIG. 2A includes the nitrite response of TGA1. FIG. 2B includes the nitrite response of TGA4. FIG. 2C shows the lack of an ammonium response of TGA1. FIG. 2D shows the lack of an ammonium response of TGA4. The asterisk (*) indicates means that significantly differ between control and treatment conditions (P<0.05).

FIG. 3 includes illustrations of the TGA1 and TGA4 effect on primary and lateral root growth in response to nitrate. FIG. 3A includes the number of initiating and emerging lateral roots of tga1/tga4 and Col-0 plants. FIG. 3B includes the primary root length measured at day 15 from Col-0, tga1, tga4, and tga1/tga4 plants, when treated with KNO₃ or KCl. Bars represent standard deviations. Different letters indicate statistically different means (P<0.05).

FIG. 4 includes illustrations of the nitrate regulation of TGA1 and TGA4 in pericycle cells. FIG. 4A includes the relative amount of TGA1 mRNA measured by RT-qPCR in pericycle cells from seedlings treated with KNO₃ or KCl for 2 hours. FIG. 4B includes the relative amount of TGA4 mRNA measured by RT-qPCR in pericycle cells from seedlings treated with KNO₃ or KCl for 2 hours. Values plotted are the mean of three replicates±standard deviation. The asterisk (*) indicates means that significantly differ between treatments (P<0.05).

FIG. 5 includes a representation of a nitrate responsive gene network controlled by TGA1/TGA4, including genes involved in N metabolism. Individual genes are displayed as triangles (transcription factors) and squares (target genes). Green lines indicate predicted transcriptional activation, while red lines indicate predicted transcriptional repression. Thin lines indicate one transcription factor binding site in the upstream region of the corresponding gene, while thick lines indicate an over-representation of the binding site. Arrows indicate positive regulation, while edges with a perpendicular line at the end indicate negative regulation. Nodes are color-coded based on function: signalling (purple); unknown genes (white); stress response (cyan); nitrogen metabolism (yellow); and other functions (grey).

FIG. 6 includes an illustration of the effects of TGA1 and TGA4 on the nitrate-dependent up-regulation of NIR, NRT2.1 and NRT2.2 genes. FIG. 6A includes NRT2.1 mRNA transcript levels in Col-0 and tga1/tga4 plants treated with KNO₃ or KCl for indicated times. FIG. 6B includes NRT2.2 mRNA transcript levels in Col-0 and tga1/tga4 plants treated with KNO₃ or KCl for indicated times. FIG. 6C includes NIR mRNA transcript levels in Col-0 and tga1/tga4 plants treated with KNO₃ or KCl for indicated times.

FIG. 7 includes illustrations of TGA1 binding to NRT2.1 and NRT2.2 promoters regions in a nitrate-dependent manner. DNAs comprising the NRT2.1 and the NRT2.2 promoter were immunoprecipitated using anti-TGA1. Non-specific IgG was used as a negative control. Immunoprecipitated promoter DNA was quantified by quantitative PCR using primers designed against the NRT2.1 and NRT2.2 promoter regions.

FIG. 8 illustrates the expression of TGA1 and TGA4 in response to nitrate is affected in chl1-5 and chl1-9 mutants. Col-0, chl1-5, chl1-9 and T101D plants were grown hydroponically with 1 mM ammonium as the only nitrogen source. At the beginning of the light period of the 15th day, plants were treated with 5 mM KNO3 or 5 mM KCl as control, for the indicated times. RNA was isolated and mRNA levels measured by RT-qPCR. The clathrin gene (At4g24550) was used as a normalization reference. (A) TGA1 transcript levels, (B) TGA4 transcript levels. Values plotted correspond to the mean of three independent biological replicates±standard deviation. The asterisk indicates means that differ significantly between mutant and wild-type plants (P<0.05).

FIG. 9 illustrates TGA1 and TGA4 expressed in lateral roots and vascular tissues of primary root. pTGA1:GUS and pTGA4:GUS lines were hydroponically grown with 1 mM ammonium as the only nitrogen source for two weeks and were treated with 5 mM KNO3 or KCl for two hours and stained for GUS activity. 5 mM KNO3 treated pTGA1:GUS (A), 5 mM KCl treated pTGA1:GUS (B), 5 mM KNO3 treated pTGA4:GUS (C), and 5 mM KCl treated pTGA4:GUS (D). Cross-section of a mature part of the primary root (E) and longitudinal section of a lateral root emerging from the primary root of a pTGA1:GUS plant treated with nitrate (G). Cross-section of a mature part of the primary root (F) and longitudinal section of a lateral and primary root from a pTGA4:GUS plant treated with nitrate (H). (Scale bars: 0.1 mm.). Numbers on (E) and (F) indicate 1: stele and 2: endodermis. Similar localization pattern was observed in eight independent transgenic lines for each genotype.

FIG. 10 illustrates TGA1 and TGA4 transcript levels for Epidermis (Epi), cortex, endodermis (Endo), pericycle (Peri) and Stele GFP-marker lines grown hydroponically for two weeks with 1 mM ammonium as the only source of N, and wherein seedlings were treated with 5 mM KNO3 or 5 mM KCl for 2 hours at the 15^{th} day.

FIG. 11 illustrates cell specific regulation by nitrate of NTR2.1, NRT2.2 and NIR genes. Epidermis (Epi), cortex, endodermis (Endo), pericycle (Peri) and Stele GFP-marker lines were grown hydroponically for two weeks with 1 mM ammonium as the only source of N. At dawn of the 15th day seedlings were treated with 5 mM KNO₃ or 5 mM KCl for 2 hours. Total RNA was isolated and mRNA levels for NRT2.1, NRT2.2 and NIR were measured using RT-qPCR. Illustrated are: (A) NRT2.1 transcript levels; (B) NRT2.2 transcript levels; and (C) NIR transcript levels.

FIG. 12 illustrates TGA1 and TGA4 regulation of the expression of NRT2.1 and NRT2.2 under treatments with low nitrate concentrations. Col-0 and tga1/tga4 plants were hydroponically grown with 1 mM ammonium as the only nitrogen source. At the beginning of the light period of the 15th day, plants were treated with 250 μM KNO₃ or 5 μM KCl as control for 2 hours. RNA was isolated and mRNA levels measured by RT-qPCR. Illustrated are: (A) NRT2.1 transcript levels; (B) NRT2.2 transcript levels; and (C) net nitrate uptake of Col-0 and tga1/tga4 plants.

FIG. 13 illustrates the nitrate response of TGA family members exposed to 5 mM KNO₃ or 5 mM KCl for the indicated times.

FIG. 14 illustrates effects on GDH2 on plants exposed to 5 mM NH₄Cl or 5 mM KCl for the indicated times.

FIG. 15 illustrates the expression of NRT2.1 in response to nitrate in chl1-5 and T101D mutants exposed to 5mM KNO₃ or 5 mM KCl for the indicated times.

FIG. 16 illustrates nitrate-dependent up-regulation of NIA1 gene mRNA levels for Col-0 and tga1/tga4 plants treated with 5 mM KNO₃ or 5 mM KCl.

### DETAILED DESCRIPTION

### I. Overview of Several Embodiments

Transcriptional regulation of nitrate uptake and assimilation genes is very important for plants, as assimilation of nitrate is an energy demanding process that must be correctly coordinated with other metabolic and physiological process for optimal plant growth in a changing environment. Disclosed herein are novel roles for the transcription factors, TGA1 and TGA4, in the response of plants to nitrate.

Using an integrative bioinformatics approach, TGA1 and TGA4 were identified as regulatory factors that mediate nitrogen responses in Arabidopsis thaliana roots. Both TGA1 and TGA4 mRNAs rapidly accumulate after nitrate and nitrite treatments in root organs. TGA1 and TGA4 are involved in nitrate-regulated primary and lateral root growth, and normal induction of NRT2.1, NRT2.2 and NIR genes by nitrate treatments, requires TGA1 and TGA4. Phenotypic analysis of tga1/tga4 double mutant plants indicated that TGA1 and TGA4 are necessary for both nitrate-dependent primary root growth and nitrate-dependent lateral root growth. Global gene expression analyses revealed that 97% of the genes with altered expression in tga1/tga4 double mutants are regulated by nitrate treatments, indicating that the TGA1 and TGA4 transcription factors have a specific role in nitrate responses in roots.

Among the nitrate-responsive genes that depend on TGA1 and TGA4 for normal nitrogen regulation of gene expression, the nitrate transporter genes, NRT2.1, NRT2.2, and the nitrite reductase (NIR) gene were identified. Specific binding of TGA1 to its cognate DNA sequence on the promoters of these target genes was confirmed by chromatin immunoprecipitation assays.

TGA factors have been implicated in plant defense against pathogen attack, stress response (Kesarwani et al. (2007) Plant Physiol. 144:336-46) and anther development (Murmu et al. (2010) Plant Physiol. 154:1492-1504). However, TGA transcription factors have not been previously associated with the nitrate response or any nutrient responses. Thus, this disclosure illuminates a new and unforeseen interaction between nitrogen and defense signaling involving TGA1 and TGA4 transcription factors.

Based on the networks analysis of transcriptomics data described in the Examples herein, none of the previously-described genes involved in the regulation of the nitrate response would be downstream of TGA1 and TGA4. See Vidal et al. (2010a) Wiley Interdiscip. Rev. Syst. Biol. Med. 2:683-93. Also, alterations of CIPK8, NLP7 and LDB37/38/39 levels have no effect on TGA1 or TGA4 expression in response to nitrate, based on previous global gene expression analyses. See Castaings et al. (2009), supra; Hu et al. (2009), supra; Rubin et al. (2009), supra. Thus, TGA1 and TGA4 likely function in a pathway to regulate nitrate and/or nitrite responses that is different and independent than those previously characterized.

### II. Abbreviations

ANOVA analysis of variance

bZIP basic leucine zipper domain

BLAST® Basic Local Alignment Search Tool

ChIP chromatin immunoprecipitation

ELISA enzyme-linked immunoSorbent assay

EMSA electrophoretic mobility shift assay

FACS fluorescence-activated cell sorting

FDR false discovery rate

NCBI National Center for Biotechnology Information

PCR polymerase chain reaction

qPCR quantitative polymerase chain reaction

RMA robust multi-array analysis

RT-PCR reverse transcription polymerase chain reaction

### III. Terms

In order to facilitate review of the various embodiments of the disclosure, the following explanations of specific terms are provided:

Endogenous: As used herein, the term “endogenous” refers to substances (e.g., nucleic acid molecules and polypeptides) that originate from within a particular organism, tissue, or cell. For example, an “endogenous” polypeptide expressed in a plant cell may refer to a polypeptide that is normally expressed in cells of the same type from non-genetically engineered plants of the same species. Likewise, an “endogenous” nucleic acid comprised in a plant cell may refer to a nucleic acid (e.g., genomic DNA) that is normally found in cells of the same type from non-genetically engineered plants of the same species. For example, a “native” or “endogenous” nucleic acid is a nucleic acid (e.g., a gene) that does not contain a nucleic acid element other than those normally present in the chromosome or other genetic material on which the nucleic acid is normally found in nature. An endogenous gene transcript is encoded by a nucleotide sequence at its natural chromosomal locus, and is not artificially supplied to the cell.

In contrast, an “exogenous” or “heterologous” molecule is a molecule that is not native to a specified system (e.g., a germplasm, variety, elite variety, and/or plant) with respect to nucleotide sequence and /or genomic location for a polynucleotide, and with respect to amino acid sequence and/or cellular localization for a polypeptide. In embodiments, exogenous or heterologous polynucleotides or polypeptides may be molecules that have been artificially supplied to a biological system (e.g., a plant cell, a plant gene, a particular plant species or variety, and/or a plant chromosome) and are not native to that particular biological system. Thus, the designation of a nucleic acid as “exogenous” may indicate that the nucleic acid originated from a source other than a naturally-occurring source, or it may indicate that the nucleic acid has a non-natural configuration, genetic location, or arrangement of elements.

Expression: As used herein, “expression” of a coding sequence (for example, a gene or a transgene) refers to the process by which the coded information of a nucleic acid transcriptional unit (including, e.g., genomic DNA or cDNA) is converted into an operational, non-operational, or structural part of a cell (e.g., a protein). Gene expression can be influenced by external signals; for example, exposure of a cell, tissue, or organism to an agent that increases or decreases expression of a gene comprised therein. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, and/or through activation, inactivation, compartmentalization, or degradation of specific protein molecules after they have been made, or by combinations of any of the foregoing. Gene expression can be measured at the RNA level or the protein level by methods known in the art, including, without limitation, Northern blot, RT-PCR, Western blot, and in vitro, in situ, or in vivo protein activity assay(s).

Increase expression: As used herein, the term “increase expression” refers to initiation of expression, as well as to a quantitative increase in the amount of an expression product produced from a template construct. In some embodiments, at least one heterologous gene may be provided to a cell or organism that otherwise comprises an endogenous copy of the same gene, so as to increase the expression of the polypeptide encoded by the gene. In such embodiments, the increase in expression may be determined by comparison of the amount of the polypeptide produced in the cell comprising the heterologous and endogenous genes, with the amount produced in the cell comprising only the endogenous gene. In some embodiments, a first polypeptide that affects transcription (e.g., TGA1 and/or TGA4) may be provided to a cell or organism, so as to increase the expression of a second polypeptide encoded by a gene under the control of the first polypeptide. In such embodiments, the increase in expression may be determined by comparison of the amount of the polypeptide produced from the gene in the presence of the first polypeptide, with the amount produced from the gene in the absence of the first polypeptide. In some embodiments, a regulatory sequence may be operably linked to a gene, so as to increase the expression of the gene. In such embodiments, the increase in expression may be determined by comparison of the amount of the polypeptide produced from the gene after operable linkage of the regulatory sequence thereto, with the amount produced from the gene before operable linkage or introduction of the regulatory sequence.

Heterologous: As used herein, the term “heterologous” refers to substances (e.g., nucleic acid molecules and polypeptides) that do not originate from within a particular organism, tissue, or cell. For example, a “heterologous” polypeptide expressed in a plant cell may refer to a polypeptide that is not normally expressed in cells of the same type from non-genetically engineered plants of the same species (e.g., a polypeptide that is expressed in different cells of the same organism or cells of a different organism).

Isolated: An “isolated” biological component (such as a nucleic acid or polypeptide) has been substantially separated, produced apart from, or purified away from other biological components in the cell of the organism in which the component naturally occurs (e.g., other chromosomal and extra-chromosomal DNA and RNA, and proteins), while effecting a chemical or functional change in the component. For example, a nucleic acid may be isolated from a chromosome by breaking chemical bonds connecting the nucleic acid to the remaining DNA in the chromosome. Nucleic acid molecules and proteins that have been “isolated” may include nucleic acid molecules and proteins purified by standard purification methods. The term embraces nucleic acids and proteins prepared by recombinant expression in a host cell, as well as chemically-synthesized nucleic acid molecules, proteins, and peptides.

Nitrogen-limiting conditions: As used herein, the term “nitrogen-limiting conditions” refers to conditions wherein there is a limited amount of nitrogen sources (e.g., nitrate and ammonium) in the soil or culture medium. The amount that is “limiting” is in some examples a range of nitrogen concentration from 0.0 to 0.2 mM; e.g., from 0 to 0.1 mM, from 0 to 0.03 mM, and from 0 to 0.05 mM.

Nucleic acid molecule: As used herein, the term “nucleic acid molecule” may refer to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A nucleic acid molecule can include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art.

Some embodiments employ a particular form of nucleic acid, an oligonucleotide (e.g., a “primer” oligonucleotide). Oligonucleotides are relatively short nucleic acid molecules, typically comprising 50 or fewer nucleobases (though some oligonucleotides may comprise more than 50). An oligonucleotide may be formed by cleavage (e.g., restriction digestion) of a longer nucleic acid comprising the oligonucleotide sequence, or it may be chemically synthesized, in a sequence-specific manner, from individual nucleoside phosphoramidites.

An oligonucleotide may be used as a probe sequence to detect a nucleic acid molecule comprising a particular nucleotide sequence. According to the foregoing, an oligonucleotide probe may be prepared synthetically or by cloning. Suitable cloning vectors are known to those of skill in the art. An oligonucleotide probe may be labeled or unlabeled. A wide variety of techniques exist for labeling nucleic acid molecules, including, for example and without limitation, radiolabeling by nick translation; random priming; and tailing with terminal deoxytransferase, where the nucleotides employed are labeled, for example, with radioactive ³²P. Other labels that may be used include, for example and without limitation: fluorophores; enzymes; enzyme substrates; enzyme cofactors; and enzyme inhibitors. Alternatively, the use of a label that provides a detectable signal, by itself or in conjunction with other reactive agents, may be replaced by ligands to which receptors bind, where the receptors are labeled (for example, by the above-indicated labels) to provide detectable signals, either by themselves, or in conjunction with other reagents. See, e.g., Leary et al. (1983) Proc. Natl. Acad. Sci. USA 80:4045-9.

Some embodiments of the invention include a polynucleotide that is “specifically hybridizable” or “specifically complementary” to a nucleotide target sequence. “Specifically hybridizable” and “specifically complementary” are terms that indicate a sufficient degree of complementarity such that stable and specific binding occurs between the polynucleotide and the nucleic acid molecule comprising the particular nucleotide target sequence. A nucleic acid molecule need not be 100% complementary to its target sequence to be specifically hybridizable. A nucleic acid molecule is specifically hybridizable when there is a sufficient degree of complementarity to avoid non-specific binding of the nucleic acid to non-target sequences under conditions where specific binding is desired, for example, under stringent hybridization conditions.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will contribute to the stringency of hybridization, though wash times also influence stringency. Calculations regarding hybridization conditions required for attaining particular degrees of stringency are known to those of ordinary skill in the art, and are discussed, for example, in Sambrook et al. (ed.) Molecular Cloning: A Laboratory Manual, 2^{nd} ed., vol. 1-3, Cold Spring Harbor Laboratory Press, Cold Spring Harbor, N.Y., 1989, chapters 9 and 11; and Hames and Higgins (eds.) Nucleic Acid Hybridization, IRL Press, Oxford, 1985. Further detailed instruction and guidance with regard to the hybridization of nucleic acids may be found, for example, in Tijssen, “Overview of principles of hybridization and the strategy of nucleic acid probe assays,” in Laboratory Techniques in Biochemistry and Molecular Biology—Hybridization with Nucleic Acid Probes, Part I, Chapter 2, Elsevier, NY, 1993; and Ausubel et al., Eds., Current Protocols in Molecular Biology, Chapter 2, Greene Publishing and Wiley-Interscience, NY, 1995.

As used herein, “stringent conditions” encompass conditions under which hybridization will only occur if there is less than 25% mismatch between the hybridization molecule and the DNA target. “Stringent conditions” include further particular levels of stringency. Thus, as used herein, “moderate stringency” conditions are those under which molecules with more than 25% sequence mismatch will not hybridize; conditions of “medium stringency” are those under which molecules with more than 15% mismatch will not hybridize; and conditions of “high stringency” are those under which sequences with more than 10% mismatch will not hybridize. Conditions of “very high stringency” are those under which sequences with more than 6% mismatch will not hybridize.

In particular embodiments, stringent conditions are hybridization overnight at 65° C. in a hybridization buffer (e.g., 6× saline-sodium citrate (SSC) buffer, 5× Denhardt's solution, 0.5% SDS, and 100 μg sheared salmon testes DNA), followed by 40 minute sequential washes at 65° C. in 0.1×SSC/0.1% SDS.

Operably linked nucleotide sequences: A first nucleotide sequence is “operably linked” with or to a second nucleotide sequence when the first nucleotide sequence is in a functional relationship with the second nucleotide sequence. For instance, a promoter is operably linked to a coding sequence if the promoter affects the transcription or expression of the coding sequence. When recombinantly produced, operably linked nucleotide sequences are generally contiguous and, where necessary to join two protein-coding regions, in the same reading frame. However, nucleotide sequences need not be contiguous to be operably linked.

The term, “operably linked,” when used in reference to a gene regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of an operably linked coding sequence. Conventional regulatory sequences include, for example and without limitation, 5′ untranslated regions; promoters; translation leader sequences; introns; enhancers; stem-loop structures; repressor binding sequences; termination sequences; and polyadenylation recognition sequences. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

Elements that may be “operably linked” to a coding sequence are not limited to promoters or other conventional regulatory sequences. For example, in some embodiments, a transcription factor polypeptide (e.g., TGA1 or TGA4) may bind to a nucleotide sequence that is upstream or downstream of a coding sequence to affect transcription of the coding sequence. In such examples, the nucleotide sequence to which the transcription factor polypeptide binds is “operably linked” to the coding sequence, even though the nucleotide sequence may not affect transcription of the coding sequence in any way in the absence of the transcription factor.

Regulatory element: As used herein, the term “regulatory element” refers to a nucleic acid molecule having gene regulatory activity; i.e., one that has the ability to affect the transcription or translation of an operably-linked transcribable nucleic acid molecule. Regulatory elements such as promoters, leaders, introns, and transcription termination regions are non-coding nucleic acid molecules having gene regulatory activity that play an integral part in the overall expression of genes in living cells. Isolated regulatory elements that function in plants are therefore useful for modifying plant phenotypes through the techniques of molecular engineering. Thus, a “regulatory element,” may be a series of nucleotides that determines if, when, and at what level a particular gene is expressed. In some examples, a regulatory element is a DNA sequence that specifically interacts with a regulatory protein, such as TGA1 and/or TGA4.

As used herein, the term “gene regulatory activity” refers to an effect exerted by a nucleic acid molecule or polypeptide on the transcription or translation of an operably linked nucleic acid molecule. An isolated nucleic acid molecule having gene regulatory activity may provide temporal or spatial expression, and/or modulate levels and rates of expression, of an operably linked nucleic acid molecule. In some examples described herein, TGA1 and/or TGA4 is provided as a polypeptide having gene regulatory activity, which increases expression of at least one nucleotide sequence that is operably linked to a regulatory DNA element that specifically binds the TGA1 and/or TGA4 polypeptide(s).

Promoter: As used herein, the term “promoter” refers to a region of DNA that may be upstream from the start of transcription, and that may be involved in recognition and binding of RNA polymerase and other proteins to effect transcription. A promoter may be operably linked to a coding sequence for expression in a cell, or a promoter may be operably linked to a nucleotide sequence encoding a signal sequence which may be operably linked to a coding sequence for expression in a cell. A “plant promoter” may be a promoter capable of initiating transcription in a plant cell.

Examples of promoters under developmental control include promoters that preferentially initiate transcription in certain tissues, for example and without limitation, leaves, roots, seeds, fibers, xylem vessels, tracheids, or sclerenchyma. Such promoters are referred to as “tissue-preferred.” Promoters which initiate transcription only in certain tissues are referred to as “tissue-specific.” A “cell type-specific” promoter primarily effects transcription in certain cell types in one or more organs, for example and without limitation, in vascular cells in roots or leaves. Exemplary tissue-specific or tissue-preferred promoters include, for example and without limitation, a root-preferred promoter (e.g., a phaseolin gene promoter); a leaf-specific and light-induced promoter, such as that from cab or rubisco; an anther-specific promoter, such as that from LAT52; a pollen-specific promoter, such as that from Zm13; and a microspore-preferred promoter, such as that from apg.

An “inducible” promoter may be a promoter that is under environmental control. See Ward et al. (1993) Plant Mol. Biol. 22:361-366. Examples of environmental conditions that may initiate transcription by inducible promoters include, for example and without limitation, anaerobic conditions and the presence of light. With an inducible promoter, the rate of transcription increases in response to an inducing agent. Exemplary inducible promoters include, but are not limited, promoters from the ACEI system that responds to copper; In2 gene promoters from maize that respond to benzenesulfonamide herbicide safeners; Tet repressor from Tn10; and the inducible promoter from a steroid hormone gene, the transcriptional activity of which may be induced by a glucocorticosteroid hormone (Schena et al. (1991) Proc. Natl. Acad. Sci. USA 88:0421).

Tissue-specific, tissue-preferred, cell type-specific, and inducible promoters constitute the class of “non-constitutive” promoters. A “constitutive” promoter is a promoter that may be active under most environmental conditions. Exemplary constitutive promoters include, for example and without limitation, plant virus promoters (e.g., the 35S promoter from cauliflower mosaic virus (CaMV)); promoters from rice actin genes; ubiquitin promoters; pEMU; MAS; maize H3 histone promoter; and the ALS promoter, Xba1/NcoI fragment 5′ to the Brassica napus ALS3 structural gene (or a nucleotide sequence similar to the Xba1/NcoI fragment) (PCT International Patent Publication No. WO 96/30530).

Any of the foregoing constitutive and non-constitutive promoters may be utilized in particular embodiments. For example, a gene to be regulated by nitrate and or nitrite in a plant cell may be provided to the plant cell wherein the gene is operably linked to a regulatory DNA element that specifically binds the TGA1 and/or TGA4 polypeptide(s) and a promoter. By way of further example, a TGA1 or TGA4 gene may be provided to a cell wherein the gene is operably linked to a constitutive or non-constitutive promoter, so as to provide expression of the TGA1 or TGA4 gene and confer attributes of the Arabidopsis nitrate response under circumstances controlled by the promoter.

Sequence identity: The term “sequence identity” (or “identity”), as used herein in the context of two nucleic acid or polypeptide sequences, may refer to the residues in the two sequences that are the same when aligned for maximum correspondence over a specified comparison window.

As used herein, the term “percentage of sequence identity” may refer to the value determined by comparing two optimally aligned sequences (e.g., nucleic acid sequences, and amino acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, for example, Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method.

As used herein with regard to nucleotide sequences, the term “substantially identical” may refer to sequences that are more than 85% identical. For example, a substantially identical nucleotide sequence may be at least 85.5%; at least 86%; at least 87%; at least 88%; at least 89%; at least 90%; at least 91%; at least 92%; at least 93%; at least 94%; at least 95%; at least 96%; at least 97%; at least 98%; at least 99%; or at least 99.5% identical to a reference sequence.

Specific binding: As used herein with regard to polypeptides and protein domains, the term “specific binding” refers to a sufficiently strong interaction between the polypeptide or protein domain and its binding partner(s) (e.g., nucleic acid(s) comprising a specific nucleotide sequence) such that stable and specific binding occurs with the binding partner(s), but not with other molecules that lack a specific amino acid sequence or specific nucleotide sequence that is recognized by the specifically-binding polypeptide. Stable and specific binding may be ascertained by techniques routine to those in the art; such as “pulldown” assays (e.g., GST pulldowns), yeast-2-hybrid assays, yeast-3-hybrid assays, ELISA, etc. Molecules that have the attribute of “specific binding” to each other may be said to “bind specifically” to each other.

Transformation: As used herein, the term “transformation” refers to the transfer of one or more nucleic acid molecule(s) into a cell. A cell is “transformed” by a nucleic acid molecule transferred into the cell when the nucleic acid molecule becomes stably replicated by the cell, either by incorporation of the nucleic acid molecule into the cellular genome, or by episomal replication. As used herein, the term “transformation” encompasses all techniques by which a nucleic acid molecule can be introduced into such a cell. Examples include, but are not limited to: transfection with viral vectors; transformation with plasmid vectors; electroporation (Fromm et al. (1986) Nature 319:791-3); lipofection (Feigner et al. (1987) Proc. Natl. Acad. Sci. USA 84:7413-7); microinjection (Mueller et al. (1978) Cell 15:579-85); Agrobacterium-mediated transfer (Fraley et al. (1983) Proc. Natl. Acad. Sci. USA 80:4803-7); direct DNA uptake; and microprojectile bombardment (Klein et al. (1987) Nature 327:70).

Transgene: An exogenous nucleic acid sequence. In some examples, a transgene may be a sequence that encodes a TGA1 or TGA4 polypeptide. In some examples, a transgene may encode a gene of interest (e.g., a reporter gene or a gene contributing to an agriculturally important plant trait) operably linked to a regulatory DNA element that specifically binds TGA1 and/or TGA4. In these and other examples, a transgene may contain one or more regulatory sequences operably linked to the transgene coding sequence. For the purposes of this disclosure, the term “transgenic,” when used to refer to an organism (e.g., a plant), refers to an organism that comprises the exogenous nucleic acid sequence. In some examples, the organism comprising the exogenous nucleic acid sequence may be an organism into which the nucleic acid sequence was introduced via molecular transformation techniques. In other examples, the organism comprising the exogenous nucleic acid sequence may be an organism into which the nucleic acid sequence was introduced by, for example, introgression or cross-pollination in a plant.

Vector: As used herein, the term “vector” refers to a nucleic acid molecule as may be introduced into a cell, for example, to produce a transformed cell. A vector may include nucleic acid sequences that permit it to replicate in a host cell, such as an origin of replication. Examples of vectors include, but are not limited to: plasmids; cosmids; bacteriophages; and viruses that carry exogenous DNA into a cell. A vector may also include one or more genes, antisense molecules, and/or selectable marker genes and other genetic elements known in the art. A vector may transduce, transform, or infect a cell, thereby causing the cell to express the nucleic acid molecules and/or proteins encoded by the vector. A vector optionally includes materials to aid in achieving entry of the nucleic acid molecule into the cell (e.g., a liposome, protein coating, etc.).

Unless specifically indicated or implied, the terms “a,” “an,” and “the” signify “at least one,” as used herein.

Unless otherwise specifically explained, all technical and scientific terms used herein have the same meaning as commonly understood by those of ordinary skill in the art to which this disclosure belongs. Definitions of common terms in molecular biology can be found in, for example, Lewin B., Genes V, Oxford University Press, 1994 (ISBN 0-19-854287-9); Kendrew et al. (eds.), The Encyclopedia of Molecular Biology, Blackwell Science Ltd., 1994 (ISBN 0-632-02182-9); and Meyers R. A. (ed.), Molecular Biology and Biotechnology: A Comprehensive Desk Reference, VCH Publishers, Inc., 1995 (ISBN 1-56081-569-8).

### IV. Nitrogen-Responsive Regulatory Factors, TGA1 and TGA4

This disclosure provides compositions and methods that exploit a new and unexpected use for the transcription factors, TGA1 and TGA4. As disclosed herein, TGA1 and TGA4 are transcription factors that influence the expression of many particular target genes in response to certain sources of nitrogen in the environment. Thus, for example, TGA1 and/or TGA4 may be used to regulate the nitrate and/or nitrite response of a plant cell, plant material, plant tissue, or plant. The properties of TGA1 and TGA4 describe herein may be used, for example, to provide transgenic plants with an altered nitrate- and nitrite-response phenotype, and to provide transgenic plants or plant cells wherein expression of a gene of interest is regulated, at least in part, by the nitrogen sources (or lack thereof) available to the plant or plant cell. For example, TGA1 and/or TGA4 may be expressed or over-expressed in a plant to initiate and/or increase primary and/or lateral root growth in the plant.

Some embodiments include a TGA1 basic leucine zipper transcription factor polypeptide. TGA1 polypeptides according to particular embodiments comprise an amino acid sequence showing increasing percentage identities when aligned with SEQ ID NO:1 (Arabidopsis thaliana TGA1). Specific amino acid sequences within these and other embodiments may comprise sequences having, for example, at least about 50%, about 55%, about 60%, about 65%, about 70%, about 75%, about 80%, 81%, 82%, 83%, 84%, 85%, 86%, 87%, 88%, 89%, 90%, 91%, 92%, 93%, 94%, 95% 96%, 97%, 98%, 99%, or 100% identity with SEQ ID NO:1. For example, some embodiments include a TGA1 polypeptide comprising an amino acid sequence selected from the group consisting of SEQ ID NO:2 (Thellungiella halophila); SEQ ID NO:3 (Arabidopsis lyrata); SEQ ID NO:4 (Brassica rapa); SEQ ID NO:5 (Arabidopsis arenosa); SEQ ID NO:6 (Vitis vinifera); SEQ ID NO:7 (Phaseolus vulgaris); SEQ ID NO:8 (Medicago truncatula); SEQ ID NO:9 (Glycine max); and SEQ ID NO:10 (Ricinus communis).

Some embodiments include a TGA4 basic leucine zipper transcription factor polypeptide. TGA4 polypeptides according to particular embodiments comprise an amino acid sequence showing increasing percentage identities when aligned with SEQ ID NO:11 (A. thaliana TGA4). Specific amino acid sequences within these and other embodiments may comprise sequences having, for example, at least about 50%, about 55%, about 60%, about 65%, about 70%, about 75%, about 80%, 81%, 82%, 83%, 84%, 85%, 86%, 87%, 88%, 89%, 90%, 91%, 92%, 93%, 94%, 95% 96%, 97%, 98%, 99%, or 100% identity with SEQ ID NO:11. For example, some embodiments include a TGA4 polypeptide comprising an amino acid sequence selected from the group consisting of SEQ ID NO:12 (M. truncatula); SEQ ID NO:13 (V. vinifera); and SEQ ID NO:14 (Zea mays).

In many embodiments, a polypeptide comprising an amino acid sequence having the aforementioned sequence identity when aligned with SEQ ID NO:1 (TGA1 polypeptides) and/or SEQ ID NO:11 (TGA4 polypeptides) is comprised within a peptide with nitrate- and nitrite-response regulatory activity, or part of a such a peptide. TGA1 polypeptides may be identified, for example, by searching a sequence database for polypeptide sequences having a threshold sequence identity with SEQ ID NO:1. TGA4 polypeptides may be identified, for example, by searching a sequence database for polypeptide sequences having a certain sequence identity with SEQ ID NO:11. Useful sequence databases may be searched by any of many methods known to those of skill in the art (e.g., utilizing NCBI's BLAST® tool). Other databases are available for many plants and other organisms through a variety of public and private commercial sources. As will be appreciated by those of skill in the art, TGA1 and TGA4 are homologous proteins, and thus, a particular polypeptide identified as comprising an amino acid sequence sharing sequence identity with SEQ ID NO:1 or SEQ ID NO:11 may also share sequence identity with the other of SEQ ID NOs:1 and 11.

Some embodiments include a nucleic acid comprising a nucleotide sequence encoding a TGA1 and/or TGA4 polypeptide, such as are described above. For example, nucleic acid sequences in some embodiments show increasing percentage identities when aligned with SEQ ID NO:15 (A. thaliana TGA1) and/or SEQ ID NO:16 (A. thaliana TGA4). Specific nucleic acid sequences within these and other embodiments may comprise sequences having, for example and without limitation, at least about 50%, about 55%, about 60%, about 65%, about 70%, about 75%, about 80%, 81%, 82%, 83%, 84%, 85%, 86%, 87%, 88%, 89%, 90%, 91%, 92%, 93%, 94%, 95% 96%, 97%, 98%, 99%, or 100% identity SEQ ID NO:15 and/or SEQ ID NO:16.

A large number of nucleic acids comprising a nucleotide sequence encoding a TGA1 and/or TGA4 polypeptide can be readily identified by those of skill in the art. For example, nucleic acid molecules may be modified without substantially changing the amino acid sequence of the encoded polypeptide, for example, by introducing permissible nucleotide substitutions according to codon degeneracy. Thus, it will be understood that any TGA1 or TGA4 polypeptide with a given amino acid sequence may be immediately reverse-engineered to any of many redundant nucleotide sequences. By way of further example, genes encoding a TGA1 or TGA4 polypeptide may be selected from any of the many available plant genomic libraries, cDNA libraries, EST libraries, and the like (e.g., by homology to SEQ ID NO:14 or SEQ ID NO:15, or by sequence similarity of an encoded polypeptide with one or more of SEQ ID NOs:1-14), or such genes may be cloned from an organism according to reliable and well-known techniques in molecular biology.

Any and all TGA1 polypeptides, TGA4 polypeptides, and nucleic acid molecules encoding either of the same find use in certain embodiments of the invention.

Some embodiments include a nucleic acid comprising a regulatory nucleotide sequence that specifically binds a TGA1 and/or TGA4 polypeptide, so as to confer nitrate and/or nitrite control upon a nucleotide sequence that is operably linked to the regulatory nucleotide sequence. In some examples, a regulatory nucleotide sequence that specifically binds a TGA1 and/or TGA4 polypeptide is comprised within an endogenous A. thaliana promoter from a gene regulated by TGA1 and/or TGA4, for example and without limitation, a gene selected from the group consisting of NRT2.1, NRT2.2, and NIR. Specific binding of a TGA1 and/or TGA4 polypeptide to a regulatory nucleotide sequence can be detected by any technique known to those of skill in the art, for example, chromatin immunoprecipitation or EMSA.

In some embodiments, nucleic acid molecules of the present invention comprise a gene regulatory element (e.g., a promoter). Promoters may be selected on the basis of the cell type into which the vector construct will be inserted. Promoters which function in bacteria, yeast, and plants are well-known in the art. The promoters may also be selected on the basis of their regulatory features. Examples of such features include enhancement of transcriptional activity, inducibility, tissue-specificity, and developmental stage-specificity. In plants, promoters that are inducible, of viral or synthetic origin, constitutively active, temporally regulated, and spatially regulated have been described. See, e.g., Poszkowski et al. (1989) EMBO J. 3:2719; Odell et al. (1985) Nature 313:810; and Chau et al. (1989) Science 244:174-81).

Useful inducible promoters include, for example, promoters induced by salicylic acid or polyacrylic acids induced by application of safeners (substituted benzenesulfonamide herbicides), heat-shock promoters, a nitrate-inducible promoter derived from the spinach nitrate reductase transcribable nucleic acid molecule sequence, hormone-inducible promoters, and light-inducible promoters associated with the small subunit of RuBP carboxylase and LHCP families.

Other useful promoters include the nopaline synthase, mannopine synthase, and octopine synthase promoters, which are carried on tumor-inducing plasmids of Agrobacterium tumefaciens; the CaMV 19S and 35S promoters; the enhanced CaMV 35S promoter; the Figwort Mosaic Virus 35S promoter; the light-inducible promoter from the small subunit of ribulose-1,5-bisphosphate carboxylase (ssRUBISCO); the EIF-4A promoter from tobacco (Mandel et al. (1995) Plant Mol. Biol. 29:995-1004); corn sucrose synthetase; corn alcohol dehydrogenase I; corn light harvesting compolex; corn heat shock protein; the chitinase promoter from Arabidopsis; the LTP (Lipid Transfer Protein) promoters; petunia chalcone isomerase; bean glycine rich protein 1; potato patatin; the ubiquitin promoter; and the actin promoter. Useful promoters particularly include root-specific promoters.

To obtain higher expression of a heterologous gene(s), it may be preferred to reengineer the gene(s) so that it is more efficiently expressed in the expression host cell (e.g., a plant cell, for example, canola, rice, tobacco, maize, cotton, and soybean). Therefore, an optional additional step in the design of a gene encoding a TGA1 and/or TGA4 polypeptide for plant expression (i.e., in addition to the provision of one or more gene regulatory elements) is reengineering of a heterologous gene protein coding region for optimal expression. Particular embodiments include a redesigned Arabidopsis gene that has been optimized to increase the expression level (i.e. produce more protein) in a transgenic plant cell from a second plant species than in a plant cell from the second plant species transformed with the original (i.e., unmodified) Arabidopsis gene sequence.

Due to the plasticity afforded by the redundancy/degeneracy of the genetic code (i.e., some amino acids are specified by more than one codon), evolution of the genomes in different organisms or classes of organisms has resulted in differential usage of synonymous codons. This “codon bias” is reflected in the mean base composition of protein coding regions. For example, organisms having genomes with relatively low G+C contents utilize more codons having A or T in the third position of synonymous codons, whereas those having higher G+C contents utilize more codons having G or C in the third position. Further, it is thought that the presence of “minor” codons within an mRNA may reduce the absolute translation rate of that mRNA, especially when the relative abundance of the charged tRNA corresponding to the minor codon is low. An extension of this reasoning is that the diminution of translation rate by individual minor codons would be at least additive for multiple minor codons. Therefore, mRNAs having high relative contents of minor codons in a particular expression host would have correspondingly low translation rates. This rate may be reflected by correspondingly low levels of the encoded protein.

In engineering optimized genes encoding a TGA1 and/or TGA4 polypeptide for expression in a plant cell (e.g., rice, tobacco, maize, cotton, and soybean), it is helpful if the codon bias of the prospective host plant(s) has been determined. Multiple publicly-available DNA sequence databases exist wherein one may find information about the codon distribution of plant genomes or the protein coding regions of various plant genes.

The codon bias is the statistical distribution of codons that the expression host uses for coding the amino acids of its proteins. The codon bias can be calculated as the frequency at which a single codon is used relative to the codons for all amino acids. Alternatively, the codon bias may be calculated as the frequency at which a single codon is used to encode a particular amino acid, relative to all the other codons for that amino acid (synonymous codons).

In designing optimized coding regions for plant expression of TGA1 and/or TGA4 polypeptides, the primary (“first choice”) codons preferred by the plant should be determined, as well as the second, third, fourth etc. choices of preferred codons when multiple choices exist. A new DNA sequence can then be designed which encodes the amino sequence of the TGA1 and/or TGA4 polypeptide, wherein the new DNA sequence differs from the native DNA sequence (encoding the polypeptide) by the substitution of expression host-preferred (first preferred, second preferred, third preferred, or fourth preferred, etc.) codons to specify the amino acid at each position within the amino acid sequence. The new sequence is then analyzed for restriction enzyme sites that might have been created by the modifications. The identified putative restriction sites are further modified by replacing these codons with a next-preferred codon to remove the restriction site. Other sites in the sequence which may affect transcription or translation of heterologous sequence are exon:intron junctions (5′ or 3′), poly-A addition signals, and/or RNA polymerase termination signals. The sequence may be further analyzed and modified to reduce the frequency of TA or CG doublets. In addition to these doublets, sequence blocks that have more than about six G or C nucleotides that are the same may also adversely affect transcription or translation of the sequence. Therefore, these blocks are advantageously modified by replacing the codons of first or second choice, etc. with the next-preferred codon of choice.

A method such as that described above enables one skilled in the art to modify gene(s) that are foreign to a particular plant so that the genes are optimally expressed in plants. The method is further illustrated in PCT International Patent Publication No. WO 97/13402 A1. Thus, optimized synthetic genes that are functionally equivalent to TGA1 and/or TGA4 genes of some embodiments may be used to transform hosts, including plants and plant cells. Furthermore, TGA1- and TGA4-encoding nucleotide sequences may also be generated, in silico, from an initial amino acid sequence. Additional guidance regarding the production of synthetic genes can be found in, for example, U.S. Pat. No. 5,380,831.

Once a TGA1- and/or TGA4-encoding nucleotide sequence has been designed on paper or in silico, actual nucleic acid molecules comprising the sequence can be synthesized in the laboratory to correspond in sequence precisely to the designed sequence. Such synthetic DNA molecules may be cloned and otherwise manipulated exactly as if they were derived from natural or native sources.

V. Mediation of Plant Nitrogen Response by TGA1 and/or TGA4

Some embodiments exploit the discovery that TGA1 and TGA4 are necessary for normal nitrate-regulated gene expression (e.g., expression of NRT2.1, NRT2.2 and NIR), and for generating plant responses to nitrate and nitrite. In particular embodiments, a TGA1 and/or TGA4 polypeptide may be expressed or over-expressed in a cell or organism, for example and without limitation, by introducing a TGA1- or TGA4-encoding nucleic acid into the cell or organism; by introducing the TGA1 and/or TGA4 polypeptide into the cell or organism; and/or by providing positive or negative signals sufficient to promote expression of the TGA1 and/or TGA4 polypeptide through an interaction of the signal(s) with regulatory elements operably linked to a TGA1- or TGA4-encoding nucleic acid in the cell or organism. In further embodiments, a TGA1 and/or TGA4 polypeptide may be knocked-out or under-expressed in a cell or organism, for example and without limitation, by disrupting, mutating, or inactivating a TGA1- and/or TGA4-encoding nucleic acid (e.g., TGA1 and/or TGA4 gene(s)); introducing an antisense nucleic acid into the cell or organism that targets a nucleic acid encoding the TGA1 and/or TGA4 polypeptide; by physically removing the TGA1 and/or TGA4 polypeptide from the cellular machinery of the cell or organism by binding the TGA1 and/or TGA4 polypeptide with antibodies or other specific binding proteins; and/or by providing positive or negative signals sufficient to reduce or eliminate expression of the TGA1 and/or TGA4 polypeptide through an interaction of the signal(s) with regulatory elements operably linked to a TGA1- or TGA4-encoding nucleic acid in the cell or organism.

In some embodiments, a TGA1 and/or TGA4 polypeptide may be expressed or over-expressed in a plant cell or organism, so as to promote the expression of one or both of nitrate transporters, NRT2.1 and NRT2.2. In further embodiments, a TGA1 and/or TGA4 polypeptide may be removed or under-expressed in a plant cell or organism, so as to decrease or eliminate the expression of one or both of nitrate transporters, NRT2.1 and NRT2.2.

Increased expression of NRT2.1 and/or NRT2.2 may be desirable for a number of reasons. In addition to its nitrate transport function, the NRT2.1 transporter serves to integrate lateral root initiation and lateral root growth. Little et al. (2005), supra; Remans et al. (2006) Plant Physiol. 140:909-21. An nrt2.1/nrt2.2 Arabidopsis mutant line showed a reduced lateral root growth in a medium supplemented with nitrate. Li et al. (2007), supra. Thus, manipulation of the levels of NRT2.1 and NRT2.2 by altering expression of TGA1 and/or TGA4, either alone or with accompanying changes in plant nutritional status may lead to altered root growth and developmental programs in plants.

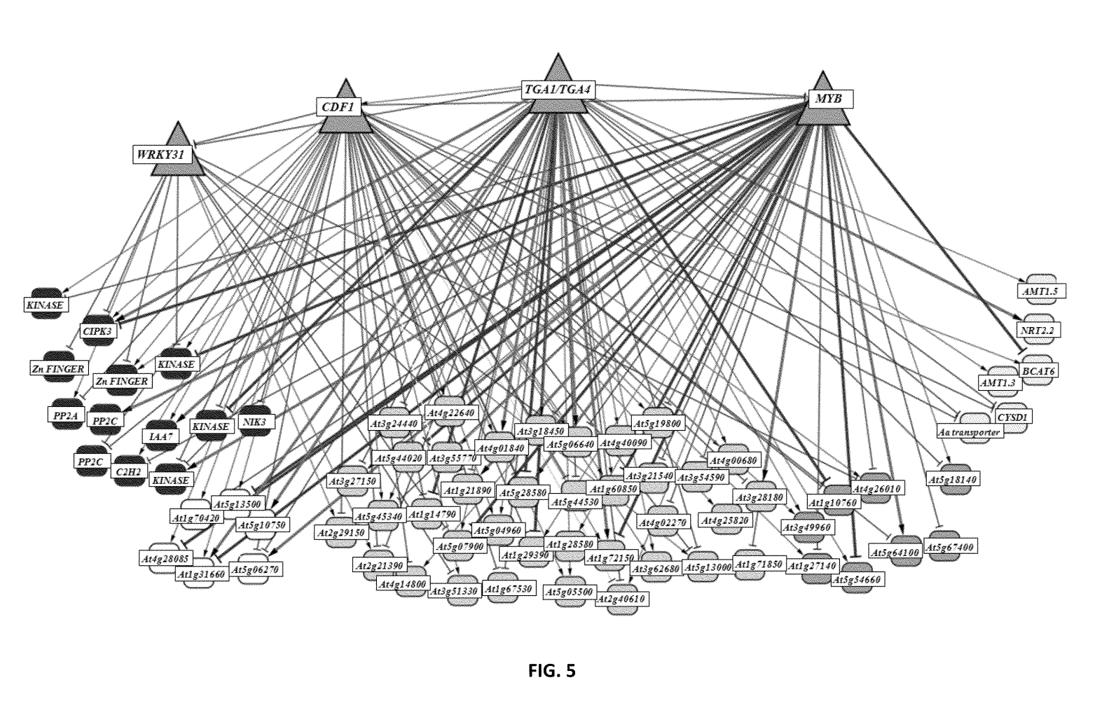

In some embodiments, a TGA1 and/or TGA4 polypeptide may be expressed or over-expressed in a plant cell or organism, so as to promote the expression of at least one other nitrogen response gene. For example, a TGA1 and/or TGA4 polypeptide may be expressed or over-expressed in a plant cell or organism, so as to promote the expression of a gene depicted in FIG. 5. In further embodiments, a TGA1 and/or TGA4 polypeptide may be removed or under-expressed in a plant cell or organism, so as to decrease or eliminate the expression of at least one other nitrogen response gene. For example, a TGA1 and/or TGA4 polypeptide may be removed or under-expressed in a plant cell or organism, so as to decrease or eliminate the expression of a gene depicted in FIG. 5.

In some embodiments, expression of TGA1 and/or TGA4 may be manipulated in a plant cell or plant, so as to affect primary and/or lateral root growth (e.g., in response to nitrate). For example, a TGA1 and/or TGA4 polypeptide may be expressed or over-expressed in a plant cell or organism, so as to stimulate and/or increase primary and/or lateral root growth. Conversely, a TGA1 and/or TGA4 polypeptide may be removed or under-expressed in a plant cell or organism, so as to eliminate and/or decrease primary and/or lateral root growth (e.g., decrease primary and/or lateral root growth in response to nitrate).

In some embodiments, expression of TGA1 and/or TGA4 may be manipulated in a plant cell or plant, so as to affect the growth of the plant cell or plant under nitrogen-limiting conditions. For example, a TGA1 and/or TGA4 polypeptide may be expressed or over-expressed in a plant cell or organism, so as to stimulate and/or increase growth of the plant under nitrogen-limiting conditions. Conversely, a TGA1 and/or TGA4 polypeptide may be removed or under-expressed in a plant cell or organism, so as to eliminate and/or decrease growth of the plant under nitrogen-limiting conditions (e.g., decrease plant growth in response to nitrate).

TGA1 can be post-translationally modified by phosphorylation (Popescu et al. (2009) Genes Dev. 23:80-92) or S-nitrosylation (Lindermayr et al. (2010) Plant Cell 22:2894-907. These and other post-translational modifications may play a role in regulation of TGA1 and/or TGA4. For example, it was recently shown that TGA1 can be S23 nitrosylated after treatments with S-nitrosoglutathione, which is a physiological nitric oxide (NO) donor. Lindermayr et al. (2010), supra. This S23 nitrosylation enhances the DNA binding activity of TGA1. Id. Because NO production is associated with NR activity (Kolbert and Erdei (2008) Plant Signal Behav. 3:972-3), and nitrite serves as a substrate for the formation of NO (Yamasaki et al. (1999) Trends Plant Sci. 4:128-9; Rockel et al. (2002) J. Exp. Bot. 53:103-10; Lea et al. (2004) Planta 219:59-65; Meyer et al. (2005) Photosynth. Res. 83:181-9; Planchet et al. (2005) Plant J. 41:732-43), a nitrate-derived metabolite (e.g., nitrite or NO) may be involved in activating TGA1 and TGA4 transcription factor activities to execute the nitrate/nitrite transcriptional response.

Thus, particular embodiments include manipulation or mimicry of the post-translational modification of TGA1 and/or TGA4, so as to influence the activity of TGA1 and/or TGA4. Moreover, upstream signaling molecules of the nitrate response pathway may be provided or removed in tandem with TGA1 and/or TGA4 expression, for example, so as to tune the effect to a desired level within the discretion of the skilled practitioner.

Without being bound to any particular theory, TGA1 and TGA4 may be part of at least two regulatory mechanisms that are activated in response to nitrate treatments. First, nitrate and/or a nitrate-derived signal (e.g., nitrite or NO) would activate TGA1 and TGA4 transcription factors to allow binding of TGA1 and TGA4 to the promoter regions of their target genes. Consequently, expression of these nitrate-responsive target genes would be increased to acclimate the cell (and plant comprising the cell) to the nitrate rich environment. Second, nitrate and/or a nitrate derived signal may also produce an induction of TGA1 and TGA4 gene expression over comparatively longer periods of time. This induction in gene expression may be part of a separate regulatory function. The timing difference of these responses may relate to the nature of the processes that are regulated (e.g., metabolic versus developmental) and/or to different spatial functions (local versus systemic). Thus, in particular embodiments, TGA1 and/or TGA4 may be manipulated in a plant or cell in a time-dependent manner, so as to achieve one or more particular desired nitrate-response(s).

VI. Plants, Plant Parts, and Plant Materials Comprising TGA1 and/or TGA4

Some embodiments are directed to a method of producing a transformed cell that comprises one or more TGA1 and/or TGA4 polypeptides (as described, supra), and or one or more nucleic acid molecule(s) comprising a nucleic acid sequence encoding a TGA1 and/or TGA4 polypeptide. Such nucleic acid molecules may also comprise, for example, non-coding regulatory elements, such as promoters. Other sequences may also be introduced into the cell along with the non-coding regulatory elements and transcribable nucleic acid molecule sequences. These other sequences may include 3′ transcriptional terminators, 3′ poly-adenylation signals, other untranslated sequences, transit or targeting sequences, selectable markers, enhancers, and operators.

A method of transformation generally comprises the steps of selecting a suitable host cell, transforming the host cell with a recombinant vector, and obtaining the transformed host cell. Technology for introduction of DNA into cells is well-known to those of skill in the art. These methods can generally be classified into five categories: (1) chemical methods (Graham and Van der Eb (1973) Virology 54(2):536-9; Zatloukal et al. (1992) Ann. N.Y. Acad. Sci. 660:136-53); (2) physical methods such as microinjection (Capechi (1980) Cell 22(2):479-88), electroporation (Wong and Neumann (1982) Biochim. Biophys. Res. Commun. 107(2):584-7; Fromm et al. (1985) Proc. Natl. Acad. Sci. USA 82(17):5824-8; U.S. Pat. No. 5,384,253), and particle acceleration (Johnston and Tang (1994) Methods Cell Biol. 43(A):353-65; Fynan et al. (1993) Proc. Natl. Acad. Sci. USA 90(24):11478-82; (3) viral vectors (Clapp (1993) Clin. Perinatol. 20(1):155-68; Lu et al. (1993) J. Exp. Med. 178(6):2089-96; Eglitis and Anderson (1988) Biotechniques 6(7):608-14); (4) receptor-mediated mechanisms (Curiel et al. (1992) Hum. Gen. Ther. 3(2):147-54; Wagner et al. (1992) Proc. Natl. Acad. Sci. USA 89(13):6099-103); and (5) bacterial-mediated mechanisms, such as with Agrobacterium. Alternatively, nucleic acids may be directly introduced into pollen by directly injecting a plant's reproductive organs. Zhou et al. (1983) Methods in Enzymology 101:433; Hess (1987) Intern. Rev. Cytol. 107:367; Luo et al. (1988) Plant Mol. Biol. Reporter 6:165; Pena et al. (1987) Nature 325:274. Other transformation methods include, for example, protoplast transformation as illustrated in U.S. Pat. No. 5,508,184. Nucleic acid molecules may also be injected into immature embryos. Neuhaus et al. (1987) Theor. Appl. Genet. 75:30.

The most commonly used methods for transformation of plant cells are: the Agrobacterium-mediated DNA transfer process (Fraley et al. (1983) Proc. Natl. Acad. Sci. USA 80:4803) (as illustrated in U.S. Pat. No. 5,824,877; U.S. Pat. No. 5,591,616; U.S. Pat. No. 5,981,840; and U.S. Pat. No. 6,384,301) and the biolistics or microprojectile bombardment-mediated process (i.e., the gene gun) (such as described in U.S. Pat. No. 5,550,318; U.S. Pat. No. 5,538,880; U.S. Pat. No. 6,160,208; U.S. Pat. No. 6,399,861; and U.S. Pat. No. 6,403,865). Typically, nuclear transformation is desired, but where it is desirable to specifically transform plastids, such as chloroplasts or amyloplasts, plant plastids may be transformed utilizing a microprojectile-mediated delivery of the desired nucleic acid molecule in certain plant species, such as for example, Arabidopsis, tobacco, potato, and Brassica species.

Agrobacterium-mediated transformation is achieved through the use of a genetically engineered soil bacterium belonging to the genus Agrobacterium. Several Agrobacterium species mediate the transfer of a specific DNA known as “T-DNA,” which can be genetically engineered to carry any desired piece of DNA into many plant species. The major events marking the process of T-DNA mediated pathogensis are: induction of virulence genes, and processing and transfer of T-DNA. This process is the subject of many reviews. See, e.g., Ream (1989) Ann. Rev. Phytopathol. 27:583-618; Howard and Citovsky (1990) Bioassays 12:103-8; Kado (1991) Crit. Rev. Plant Sci. 10:1-32; Zambryski (1992) Annual Rev. Plant Physiol. Plant Mol. Biol. 43:465-90; Gelvin (1993) in Transgenic Plants, Kung and Wu eds., Academic Press, San Diego, Calif., pp. 49-87; Binns and Howitz (1994) In Bacterical Pathogenesis of Plants and Animals, Dang, ed., Berlin: Springer Verlag., pp. 119-38; Hooykaas and Beijersbergen (1994) Ann. Rev. Phytopathol. 32:157-79; Lessl and Lanka (1994) Cell 77:321-4; and Zupan and Zambryski (1995) Annual Rev. Phytopathol. 27:583-618.

To select or score for transformed plant cells regardless of transformation methodology, the DNA introduced into the cell may contain a gene that functions in a regenerable plant tissue to produce a compound that confers upon the plant tissue resistance to an otherwise toxic compound. Genes of interest for use as a selectable, screenable, or scorable marker include, but are not limited to, β-glucuronidase (GUS), green fluorescent protein (GFP), luciferase, and antibiotic or herbicide tolerance genes. Examples of antibiotic resistance genes include genes conferring resistance to the penicillins, kanamycin (and neomycin, G418, bleomycin); methotrexate (and trimethoprim); chloramphenicol; and tetracycline. For example, glyphosate resistance may be conferred by a herbicide resistance gene. Della-Cioppa et al. (1987) Bio/Technology 5:579-84. Other selection devices can also be implemented, including for example and without limitation, tolerance to phosphinothricin, bialaphos, and positive selection mechanisms (Joersbro et al. (1998) Mol. Breed. 4:111-7), and are considered within the scope of embodiments of the present invention.

The transformed cells, identified by selection or screening and cultured in an appropriate medium that supports regeneration, may then be allowed to mature into plants.

The presently disclosed methods may be used with any transformable plant cell or tissue. Transformable cells and tissues, as used herein, includes but is not limited to those cells or tissues that are capable of further propagation to give rise to a plant. Those of skill in the art recognize that a number of plant cells or tissues are transformable in which after insertion of exogenous DNA and appropriate culture conditions the plant cells or tissues can form into a differentiated plant. Tissue suitable for these purposes can include but is not limited to immature embryos, scutellar tissue, suspension cell cultures, immature inflorescence, shoot meristem, nodal explants, callus tissue, hypocotyl tissue, cotyledons, roots, and leaves.

The regeneration, development, and cultivation of plants from transformed plant protoplast or explants are known in the art. Weissbach and Weissbach (1988) Methods for Plant Molecular Biology, (Eds.) Academic Press, Inc., San Diego, Calif.; Horsch et al. (1985) Science 227:1229-31. This regeneration and growth process typically includes the steps of selecting transformed cells and culturing those cells through the usual stages of embryonic development through the rooted plantlet stage. Transgenic embryos and seeds are similarly regenerated. In this method, transformants are generally cultured in the presence of a selective media which selects for the successfully transformed cells and induces the regeneration of plant shoots. Fraley et al. (1993) Proc. Natl. Acad. Sci. USA 80:4803. These shoots are typically obtained within two to four months. The resulting transgenic rooted shoots are thereafter planted in an appropriate plant growth medium such as soil. Cells that survive the exposure to a selective agent, or cells that have been scored positive in a screening assay, may be cultured in media that supports regeneration of plants. The shoots may then be transferred to an appropriate root-inducing medium containing the selective agent and an antibiotic to prevent bacterial growth. Many of the shoots will develop roots. These are then transplanted to soil or other media to allow the continued development of roots. The method, as outlined above, will generally vary depending on the particular plant strain employed, and particulars of the methodology are therefore within the discretion of one of skill in the art.

The regenerated transgenic plants may be self-pollinated to provide homozygous transgenic plants. Alternatively, pollen obtained from the regenerated transgenic plants may be crossed with non-transgenic plants, preferably inbred lines of agronomically important species. Conversely, pollen from non-transgenic plants may be used to pollinate the regenerated transgenic plants.

The transgenic plant may pass along the transformed nucleic acid sequence to its progeny. The transgenic plant is preferably homozygous for the transformed nucleic acid sequence and transmits that sequence to all of its offspring upon, and as a result of, sexual reproduction. Progeny may be grown from seeds produced by the transgenic plant. These additional plants may then be self-pollinated to generate a true breeding line of plants.

The progeny from these plants may be evaluated, among other things, for gene expression. The gene expression may be detected by several common methods such as western blotting, northern blotting, immunoprecipitation, and ELISA. The transformed plants may also be analyzed for the presence of the introduced DNA and the expression level and/or fatty acid profile conferred by the nucleic acid molecules and amino acid molecules of the present invention. Those of skill in the art are aware of the numerous methods available for the analysis of transformed plants. For example, methods for plant analysis include, but are not limited to, Southern blots or northern blots, PCR-based approaches, biochemical assays, phenotypic screening methods, field evaluations, and immunodiagnostic assays.

Methods for specifically transforming dicots are well-known to those skilled in the art. Transformation and plant regeneration using these methods have been described for a number of crops including, but not limited to, members of the genus Arabidopsis, cotton (Gossypium hirsutum), soybean (Glycine max), peanut (Arachis hypogaea), and members of the genus Brassica. Methods for transforming dicots, primarily by use of Agrobacterium tumefaciens, and obtaining transgenic plants have been published for cotton (U.S. Pat. No. 5,004,863; U.S. Pat. No. 5,159,135; U.S. Pat. No. 5,518,908); soybean (U.S. Pat. No. 5,569,834; U.S. Pat. No. 5,416,011; McCabe et al. (1988) Biotechnology 6:923; Christou et al. (1988) Plant Physiol. 87:671-4); Brassica (U.S. Pat. No. 5,463,174); peanut (Cheng et al. (1996) Plant Cell Rep. 15:653-7; McKently et al. (1995) Plant Cell Rep. 14:699-703); papaya; and pea (Grant et al. (1995) Plant Cell Rep. 15:254-8).

Methods for transforming monocots are also well-known in the art. Transformation and plant regeneration using these methods have been described for a number of crops including, but not limited to, barley (Hordeum vulgarae); maize (Zea mays); oats (Avena sativa); orchard grass (Dactylis glomerata); rice (Oryza sativa, including indica and japonica varieties); sorghum (Sorghum bicolor); sugar cane (Saccharum sp); tall fescue (Festuca arundinacea); turfgrass species (e.g., Agrostis stolonifera, Poa pratensis, Stenotaphrum secundatum); wheat (Triticum aestivum); and alfalfa (Medicago sativa). It is apparent to those of skill in the art that a number of transformation methodologies can be used and modified for production of stable transgenic plants for any number of target crops of interest.

Any plant may be chosen for use in the presently disclosed methods. Preferred plants for modification according to the present invention include, for example and without limitation, oilseed plants, Arabidopsis sp. (e.g., A. thaliana), borage (Borago spp.), canola (Brassica spp.), castor (Ricinus communis), cocoa bean (Theobroma cacao), corn (Zea mays), cotton (Gossypium spp), Crambe spp., Cuphea spp., flax (Linum spp.), Lesquerella and Limnanthes spp., Linola, nasturtium (Tropaeolum spp.), Oenothera spp., olive (Olea spp.), palm (Elaeis spp.), peanut (Arachis spp.), rapeseed, safflower (Carthamus spp.), soybean (Glycine and Soja spp.), sunflower (Helianthus spp.), tobacco (Nicotiana spp.), Vernonia spp., wheat (Triticum spp.), barley (Hordeum spp.), rice (Oryza spp.), oat (Avena spp.) sorghum (Sorghum spp.), and rye (Secale spp.) or other members of the Gramineae.

It is apparent to those of skill in the art that a number of transformation methodologies can be used and modified for production of stable transgenic plants from any number of target crops of interest.

Each document, patent, and reference cited herein is herein incorporated by its entirety.

The following examples are provided to illustrate certain particular features and/or embodiments. These examples should not be construed to limit the invention to the particular features or embodiments described.

### EXAMPLES

### Example I

### Materials and Methods

Bioinformatics analyses to predict nitrate regulatory genes: Bioinformatics analyses were performed using a network model of plant gene interactions to identify nitrate regulatory genes. In order to enrich the model's predictions, available microarray expression data corresponding to nitrate treatments were used. Wang et al. (2003), supra; Scheible et al. (2004), supra; Wang et al. (2004), supra; Gutierrez et al. (2007), supra.

First, all Arabidopsis transcription factor genes were selected. Second, those genes that were significantly regulated by nitrate were selected. Third, a rank score was assigned to the genes based on the magnitude (fold-change) observed when comparing treatment and control experiments in each of the microarray analyses. Fourth, a rank score was assigned to the genes based on the number of connections observed in the network model. Gutierrez et al. (2007), supra. Highly connected genes may be “regulatory hubs.” Barabasi and Oltvai (2004) Nat. Rev. Genet. 5:101-13. Fifth, we assigned a rank score to the genes based on the size of the gene family. Gene family sizes were determined using BLASTCLUST™ using the method of Gutierrez et al. (2004) Genome Biol. 5:R53. This last criterion was used to reduce the chance of a lack of phenotype in the corresponding mutants due to functional redundancy. Finally, the median of all the independently obtained score ranks was calculated and ordered, thereby providing the final list of genes.

Plant Material and Growth Conditions: Wild-type Arabidopsis thaliana Columbia-0 (“Col-0”) was used in all experiments. All mutants utilized were also in the Col-0 background. tga1, tga4 single mutants and tga1/tga4 double mutant plants were kindly donated by Dr. Xinnian Dong, Duke University, North Carolina, USA. Kesarwani et al. (2007), supra. Nitrate reductase (NR)-NULL mutant lines were kindly provided by Nigel Crawford, University of California San Diego, La Jolla, Calif. Wang et al. (2004), supra. The source of the GFP line used that marks pericycle (E374) are available from GFP enhancer trap lines from the University of Pennsylvania.

Plants were growth in hydroponic cultures using MS31 modified basal salt media without nitrogen (Phytotechnology Laboratories). This medium was supplemented with 0.5 mM ammonium succinate and 3 mM sucrose. After 14 days under long-day (16/8-h light/dark) conditions at 22° C. (in Percival incubators), plants were treated for the indicated period of time at the beginning of the light cycle on day 15 with 5 mM KNO₃ or 5 mM KCl as a control. For the phenotypic analysis of the root response to nitrate treatments, seedlings were grown as described above, and were treated with 5 mM KNO₃ or 5 mM KCl (as negative control) for 3 days. For primary root measurements, plant images were acquired using an EPSON™ Perfection V700 Photo scanner, and roots were measured using the IMAGEJ™ program. Lateral roots were counted using DIC optics on a NIKON™ Eclipse 80i microscope.

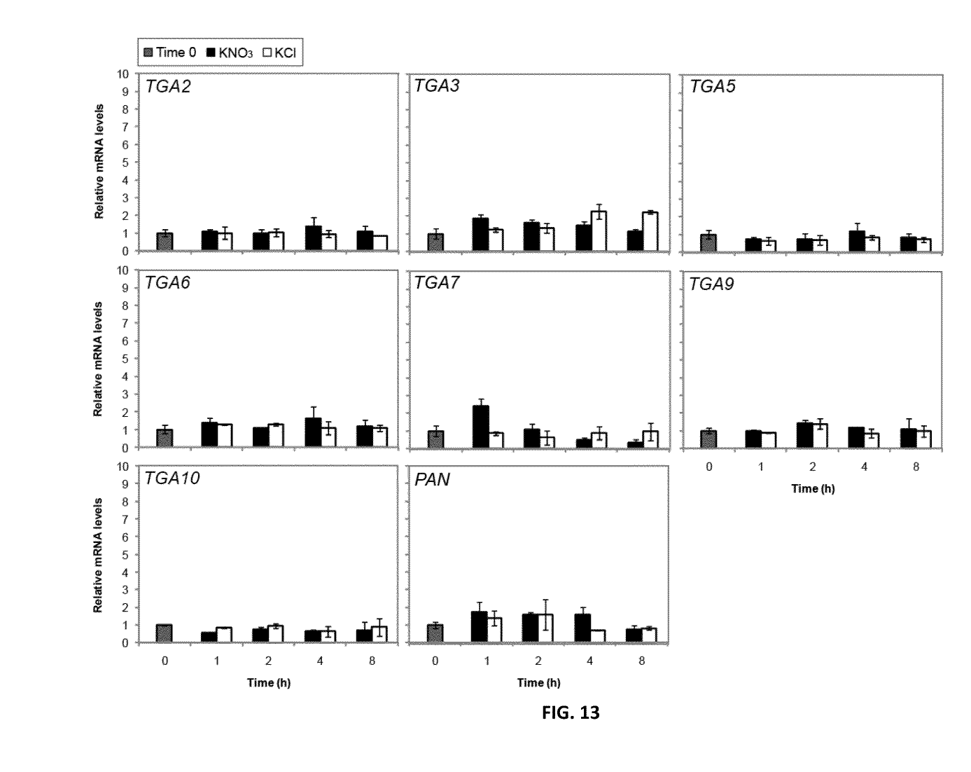

RNA Isolation and RT-qPCR: RNA was isolated from whole roots with the TRIZOL® reagent according to the instructions of the manufacturer (Invitrogen). cDNA synthesis was carried out using the ImProm-II™ reverse transcriptase according to the instruction of the manufacturer (Promega). RT-qPCR was carried out using the Brilliant SYBR® Green QPCR Reagents on a Stratagene MX3000P qPCR system. RNA levels were normalized relative to clathrin (Atg4g24550). As shown in FIG. 13, values plotted correspond to the mean of three biological replicates±standard deviation; no statistically different means were found (p<0.05).

Protoplast Generation and Cell Sorting of Pericycle Cells: Enhancer Trap line E374 seedlings marking pericycle were grown under the same experimental conditions set forth above. Plants grown hydroponically with 0.5 mM ammonium succinate as the only nitrogen source were treated on the onset of day 15 with 5 mM KNO₃ or 5 mM KCl for 2 hrs. Roots were harvested, and protoplasts were generated by treating with cellulase and pectolyase according to the methods of Birnbaum et al. (2005) Nat. Methods 2:615-9; and Gifford et al. (2008) Proc. Natl. Acad. Sci. USA 105:803-8. GFP24-expressing lines were isolated using a FACS and collected directly in the lysis buffer from the mirVana™ total RNA extraction kit (Ambion, 1560M). cDNA synthesis and gene expression analyses were performed as set forth above.

Gene expression and network analysis: cDNA synthesis, array hybridization, and normalization of signal intensities were performed according to the instructions provided by Affymetrix. Data was normalized in the R software (Affymetrix) using robust multi-array analysis (RMA). Irizarry et al. (2003) Biostatistics 4:249-64. Normalized data was subjected to a two-way ANOVA analysis (P<0.05) with a false discovery rate of 5%. For the ANOVA analysis, a model was used considering the expression of a given gene Y as:

Yᵢ=β₀T+β₁G+β₂TG+ε, where Eq. 1

β₀ is the global mean; β₁, β₂ and β₃ are the effects of the treatment, genotype, and the interaction between these two factors, respectively; and ε is the unexplained variance.

A molecular network for genes possessing a significant Treatment:Genotype interaction factor was created using the “Gene networks” tool available through the VirtualPlant™ (virtualplant.org). Protein-DNA interactions were included, considering at least one transcription factor binding site in the upstream gene region and over-representation of the transcription factor binding site (two standard deviations) above the mean occurrence in all the upstream sequence in the genome. To improve the regulatory interaction predictions, the protein-DNA interactions were filtered to include only transcription factor/target pairs whose expression values significantly correlated (P<0.05) in our microarray experiments. The resulting network was visualized using Cytoscape™ software. Shannon et al. (2003) Genome Res. 13:2498-504.

Chromatin immunoprecipitation (ChIP) assays: ChIP assays were performed according to the method of Saleh et al. (2008) Nat. Protoc. 3:1018-25. Briefly, plants grown hydroponically for two weeks with 0.5 mM ammonium succinate as the only nitrogen source were treated with 5 mM KNO₃ or 5mM KCl as control at dawn of day 15 (beginning of the light period). Roots were collected and immediately fixed in 1% formaldehyde for 10 min under vacuum at room temperature. Cross-linking was stopped by the addition of glycine to a final concentration of 0.125 M.

Nuclei were prepared for chromatin isolation: Isolated chromatin was sonicated 22 times for 15 sec each at 1 cycle and 40% amplitude (Dr. Hielscher GmbH Bioruptor). A small aliquot of sheared chromatin was removed to serve as a control (Input). The diluted chromatin was used for IP with the anti-TGA1 antibody and an unspecific IgG used as negative control. Immunoprecipitated DNA was amplified by quantitative PCR using the following sets of primers: AtNRT2.1 (forward, 5′-CTATCCTGTATCACTGTATGTAACCAG (SEQ ID NO:17); reverse, 5′-GGATGGATAGTCAACAATATGGTTGTG (SEQ ID NO:18)) and AtNRT2.2 (forward, 5′-CTCAACAGAGGGAACACCGG (SEQ ID NO:19); reverse, 5′-CCCAAAATATATTACAATGTAGTTG (SEQ ID NO:20)).

A ranking system was developed to integrate diverse data types, which system identified TGA1 and TGA4 as potentially important regulatory factors controlling nitrogen responses in Arabidopsis. We experimentally demonstrated the importance of TGA1 and TGA4 as important nitrogen response regulators, and further demonstrated that TGA1 and TGA4 mediate nitrogen regulation of important genes involved in nitrate uptake and reduction. It was also determined that TGA1 and TGA4 are important regulatory factors for both primary and lateral root growth in response to nitrate. These results identify the TGA1 and TGA4 transcription factors as important regulatory factors in the plant root nitrogen response.

### Example II

### Determination of Nitrate Response Regulators in Arabidopsis

Transcription factors were ranked in each experiment based on the absolute response to nitrate treatments (best rank for strongest response, induced or repressed) according to the methodology set forth in Example I. The ranks for each experiment were averaged to generate one score for nitrate regulation. The top candidate of the analysis was TGA1 (At5g65210), a bZIP transcription factor that has not been previously associated with the nitrate response. TGA4 (At5g10030), a closely related member of the bZIP family, was also found in the ranking with a lower score. Because of their reported functional redundancy (Kesarwani et al. (2007), supra), both TGA1 and TG4 were selected for further analysis.

### Example III

### Nitrate Regulates the Expression of TGA1 and TGA4

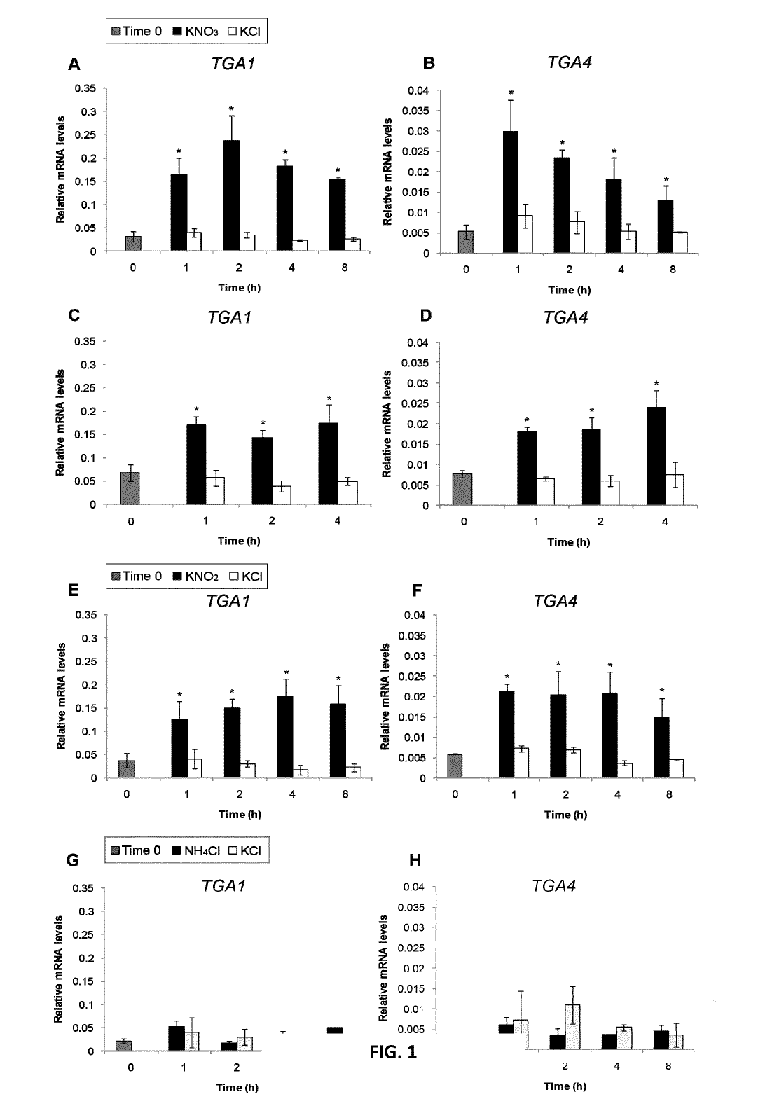

As a first step to analyze the possible role of these transcription factors in the nitrate response, TGA1 and TGA4 mRNA levels were measured in time-course experiments after nitrate treatments. Wild-type Col-0 plants were grown hydroponically for two weeks with 0.5 mM ammonium succinate as the only nitrogen source. At the beginning of the light period of day 15, plants were exposed to 5 mM KNO₃ or KCl (control). Root organs were harvested for RNA isolation 1, 2, 4, and 8 hours thereafter. Transcript levels of TGA1 and TGA4 were measured using quantitative RT-qPCR, and the clathrin gene was used as a reference standard. mRNA levels are relative to time 0. FIG. 1(A-B). As shown in FIGS. 1A and 1B, both TGA1 and TGA4 mRNA accumulated quickly after KNO₃, but not after KCl treatments, indicating that expression of these genes is regulated by nitrate treatments in roots. In order to evaluate whether the nitrate regulation of TGA1 and TGA4 was common to all TGA family members (Jakoby et al. (2002) Trends Plant Sci. 7:106-11), mRNA levels were measured for TGA2, TGA3, TGA5, TGA6, TGA7, TGA9, TGA10 and PAN. Under the same experimental conditions set forth above, nitrate treatments did not affect the expression of these other TGA transcription factors, as shown in FIG. 13. Under the same experimental conditions, nitrate treatments did not affect the expression of other TGA transcription factors. These results indicate that nitrate treatments specifically affect TGA1 and TGA4 expression in roots.

### Example IV

### Nitrate Metabolites Regulate the Expression of TGA1 and TGA4

To evaluate whether the observed regulation was due to nitrate directly, or to N-metabolites produced after nitrate reduction, similar experiments were carried out in a NR-null mutant. Wang et al. (2004), supra. Plants were grown in hydroponic media with ammonium as the only nitrogen source. At the beginning of the light period of day 15, roots were harvested (time 0) or exposed to 250 mM KNO₃, 250 mM KCl, 5 mM NH₄Cl, or 5 mM KCl for the indicated times. Roots were harvested, and total RNA was isolated for RT-qPCR analysis, and the clathrin gene was used for normalization of the RNA levels. FIGS. 1C and 1D.

The lack of NR activity in the NR-null mutant prevents nitrate reduction, blocking the production of downstream signals. Wang et al. (2004), supra. Consequently, genes that respond to nitrate in both wild-type and the NR-null mutant are regulated directly by nitrate. In the NR-null mutant, both TGA1 and TGA4 mRNA levels were induced by nitrate treatments after 1 hour. FIGS. 1C and 1D. However, accumulation of TGA1 and TGA4 mRNAs after nitrate treatment was significantly reduced in the NR-null mutant, as compared with wild-type plants. Albeit severely reduced, the detected increase in TGA1 and TGA4 mRNA levels in the NR-null mutant plants after nitrate treatments indicates regulation of expression of these genes by nitrate and other N metabolites.

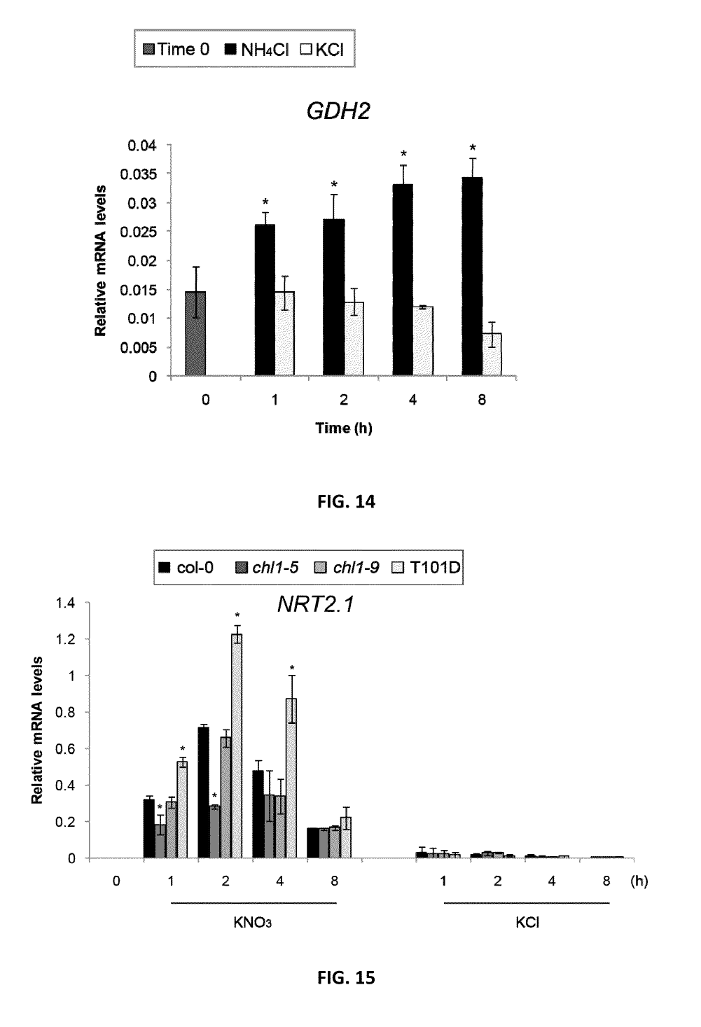

To identify additional N metabolic signals that contribute to TGA1 and TGA4 regulation, we evaluated TGA1 and TGA4 mRNA levels over time after nitrite or ammonium treatments. Previous studies showed that 250 μM nitrite is the optimal concentration to obtain peak of induction of nitrite-responsive genes (Wang et al., 2007). 250 μM nitrite treatments induced both TGA1 and TGA4 transcript levels (FIGS. 1E and 1F). No significant changes in mRNA levels were observed for these genes after ammonium treatments (FIGS. 1G and 1H) using reported conditions to evaluate ammonium regulation of GDH2 and other ammonium-responsive genes (Patterson et al., 2010) (FIG. 14). These results indicate TGA1 and TGA4 are induced by nitrate and nitrite in Arabidopsis roots.

### Example V

### Nitrite Regulates the Expression of TGA1 and TGA4

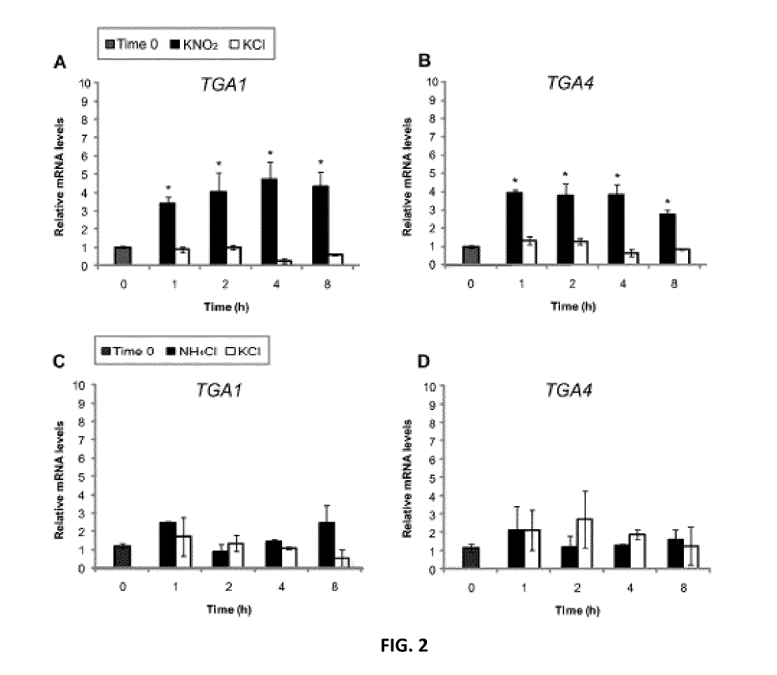

To identify additional nitrogen metabolic signals that contribute to TGA1 and TGA4 regulation, TGA1 and TGA4 mRNA levels were evaluated over time after nitrite or ammonium treatments. Nitrite treatments induced both TGA1 and TGA4 transcript levels. FIG. 2(A-B). However, no significant change in the level of either mRNA was observed for TGA1 and TGA4 after ammonium treatments. FIG. 2(C-D). These results indicate that TGA1 and TGA4 are induced by nitrate and nitrite in roots, suggesting that these transcription factors may be involved in the regulation of initial N-metabolism steps of both nitrate uptake and reduction.

### Example VI

### TGA1 and TGA4 Promote Primary and Lateral Root Growth

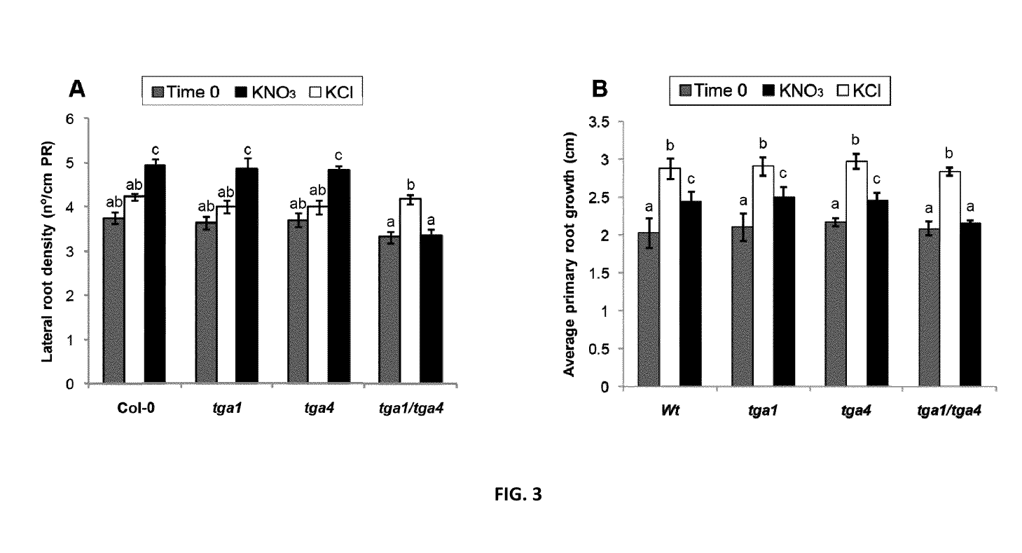

To evaluate the impact of TGA1 and TGA4 on root growth and development, the responses to 3 days KNO₃ or KCl (control) treatment of tga1 and tga4 single mutants and tga1/tga4 double mutant plants (Kesarwani et al. (2007), supra) were analyzed. The primary root length was measured in hydroponically grown plants for two weeks, under the same experimental conditions set forth above, and after 3 days of 5 mM KNO₃ or KCl treatment. Specifically, plants were grown hydroponically for two weeks with 0.5 mM ammonium succinate as the only source of nitrogen. At dawn of day 15, seedlings were treated with 5 mM KNO₃ or 5 mM KCl for 3 days. Primary root lengths under these conditions from Col-0, tga1, tga4 and tga1/tga4 plants were measured as described in Example I. FIG. 3.

Both tga1 and tga4 single mutants showed normal primary root growth as compared to wild-type plants under both KNO₃ and KCl treatments. FIG. 3A. The lack of phenotypes in the single mutant lines was consistent with the high sequence similarity (Xiang et al. (1997) Plant Mol. Biol. 34:403-15) between these two genes and their previously reported functional redundancy within the context of pathogenic response regulation (Kesarwani et al. (2007), supra).

In contrast to the single mutants, the tga1/tga4 double mutant showed reduced primary root growth as compared to wild-type plants under the KNO₃ treatment, but not under the KCl control treatment. FIG. 3A. Furthermore, the lateral root density was evaluated in response to nitrate under the same experimental conditions, and nitrate treatments increased lateral roots density in wild-type (Col-0) plants comprising TGA1 and TGA4 alleles. However, tga1/tga4 double mutant plants showed an altered lateral root response, displaying a decreased lateral roots density as compared with wild-type plants in nitrate treatments. FIG. 3B.

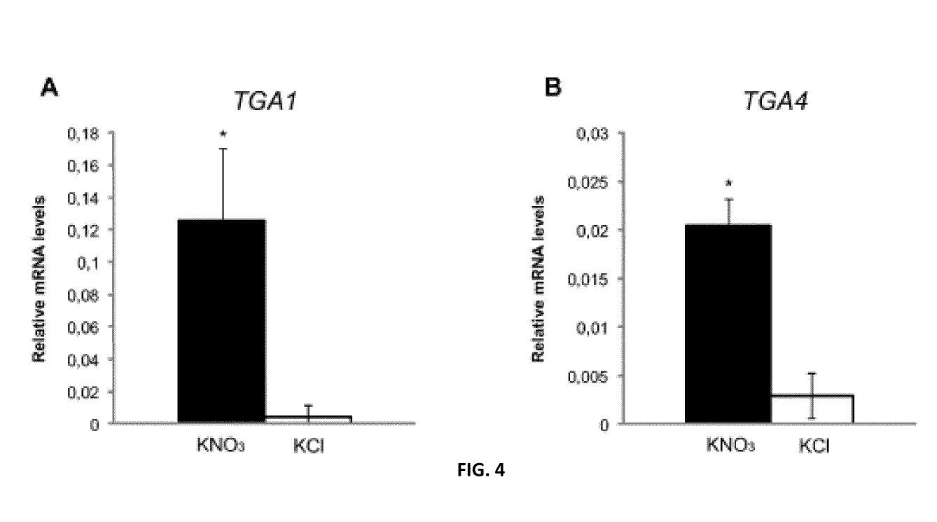

Pericycle is the outermost part of the stele, and lateral roots are initiated from pericycle tissue. Dolan et al. (1993) Development 119:71-84; Malamy and Benfey (1997) Development 124:33-44. In order to evaluate whether nitrate treatments regulate the expression of TGA1 and TGA4 in the pericycle cell layer, pericycle marker line plants were grown hydroponically for two weeks with 0.5 mM ammonium succinate as the only source of nitrogen. At dawn of day 15, seedlings were treated with 5 mM KNO₃ or 5 mM KCl for 2 hours. Protoplasts were prepared from roots, and pericycle cells expressing GFP were sorted by FACS. Total RNA was isolated from the pericycle cells, and mRNA levels for TGA1 and TGA4 were measured using RT-qPCR. TGA1 and TGA4 mRNA were found to accumulate after KNO₃ treatments, but not after KCl treatments, in pericycle cells. FIG. 4. This result indicates that TGA1 and TGA4 expression is regulated by nitrate treatments in pericycle cells, where lateral root initiation occurs. These results indicate that TGA1 and TGA4 are important for modulating root system architecture in response to nitrate.

### Example VII

### A Nitrate Responsive Gene Network Controlled by TGA1 and TGA4

In order to identify TGA1 and TGA4 target genes that may underlie the role of these transcription factors in primary and lateral root growth in the presence of nitrogen, we performed transcriptomics analyses to evaluate the effect of nitrate in roots of wild-type and tga1/tga4 double mutant plants using an Arabidopsis gene chip (ATH1; Affymetrix). Plants were grown in MS medium with ammonium succinate as the only nitrogen source, and were treated with 5 mM KNO₃ or KCl for two hours as described above. Total RNA was isolated from root organs and prepared for gene chip hybridization as described in Example I. Gene expression data was normalized using RMA, and differential gene expression was determined using two-way ANOVA according to the methods of Krouk et al. (2009) PLoS Comput. Biol. 5:e1000326. The factors considered for the ANOVA models were plant genotype (G), the treatment (T), and the interaction between genotype and treatment (TG), and a 5% FDR was used to define significant changes in gene expression. The results indicated that 827 genes are regulated by the nitrate treatments (T) under our experimental conditions. The number and nature of the genes regulated by nitrate in these experiments was comparable to what has been previously reported for genome-wide analysis of the Arabidopsis nitrate response. Wang et al. (2003), supra; Scheible et al. (2004), supra; Wang et al. (2004), supra.

96 genes were identified in which the TG factor was significant. These 96 genes correspond to genes whose response to nitrate is altered in the tga1/tga4 double mutant as compared to wild-type TGA1/TGA4 plants. Only four genes showed genotype as the only significant factor in the model, indicating that the effect of the tga1/tga4 mutation is most notorious in the context of the nitrate response. Globally, 15% of the genes showed altered regulation of expression in response to nitrate in the tga1/tga4 double mutant. Furthermore, 97% of the genes with altered gene expression in the tga1/tga4 double mutant were also regulated by nitrate. This result strongly associates TGA1 and TGA4 with specific aspects of the nitrate response.

To uncover regulatory interactions of genes whose response to nitrate depends on TGA1 and TGA4, a network view of genes that present a significant TG factor was generated using the Gene Networks tools available through the VirtualPlant™ website. Katari et al. (2010) Plant Physiol. 152:500-15. Cytoscape™ was used to visualize the resulting networks, where genes are represented as nodes connected by edges showing regulatory interactions. FIG. 5. According to the network, both TGA1 and TGA4 positively regulate the expression of the nitrate transporter, NRT2.2. NRT2.2 is important for nitrate uptake in Arabidopsis. Li et al. (2007), supra. In addition, genes involved in other signaling pathways were observed in the network, for example, SERINE/THREONINE PROTEIN PHOSPHATASE 2A (PP2A, At5g25510); PROTEIN PHOSPHATASE 2C (PP2C, At4g38520); a CBL-INTERACTING PROTEIN KINASE 3 (CIPK3, At2g26980); and an AUXIN/INDOLE-3-ACETIC ACID 7 (IAA7, At3g23050) transcription factor. Some genes participating in stress responses, such as peroxidases, were also found to be regulated by TGA1 and TGA4.

These results indicate that in response to nitrate treatments, TGA1 and TGA4 regulate the expression of target genes involved in nitrate uptake, as well as in cell signaling, and stress responses. Altered regulation of the expression of such target genes in response to a nitrate treatment in the tga1/tga4 double mutant may explain the altered phenotypes observed.

### Example VIII

### Regulatory Role of TGA1 and TGA4 in the Nitrate Response

Our data predict that a gene participating directly in nitrate uptake is a target of TGA1 and TGA4 in the nitrate response. In order to determine if the expression of genes participating in nitrate uptake and reduction is affected in the tga1/tga4 double mutant, mRNA levels of the known nitrate-responsive genes, NRT2.1, NRT2.2, NIA1 and NIR, were measured using RT-qPCR after nitrate treatments in wild-type and tga1/tga4 mutant plants. Specifically, Col-0 and tga1/tga4 plants were grown in a hydroponics system with ammonium as the only nitrogen source. At the beginning of the light period of day 15, plants were treated with 5 mM KNO₃ or 5 mM KCl (control), for the indicated times. RNA was isolated, and mRNA levels were measured by RT-qPCR, where the clathrin gene was used for normalization.

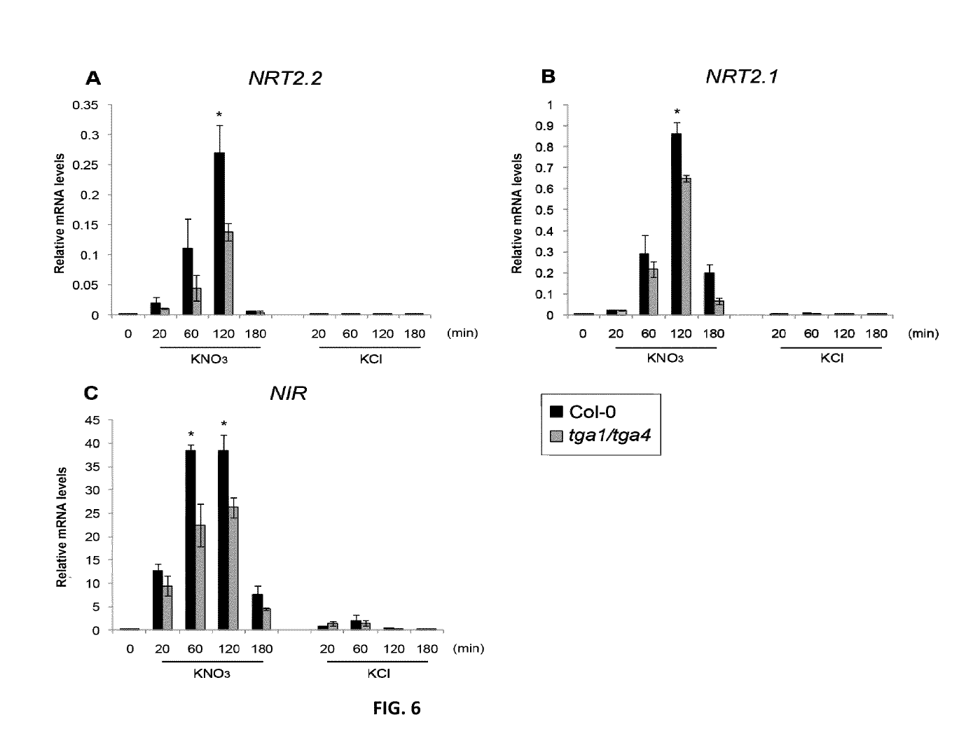

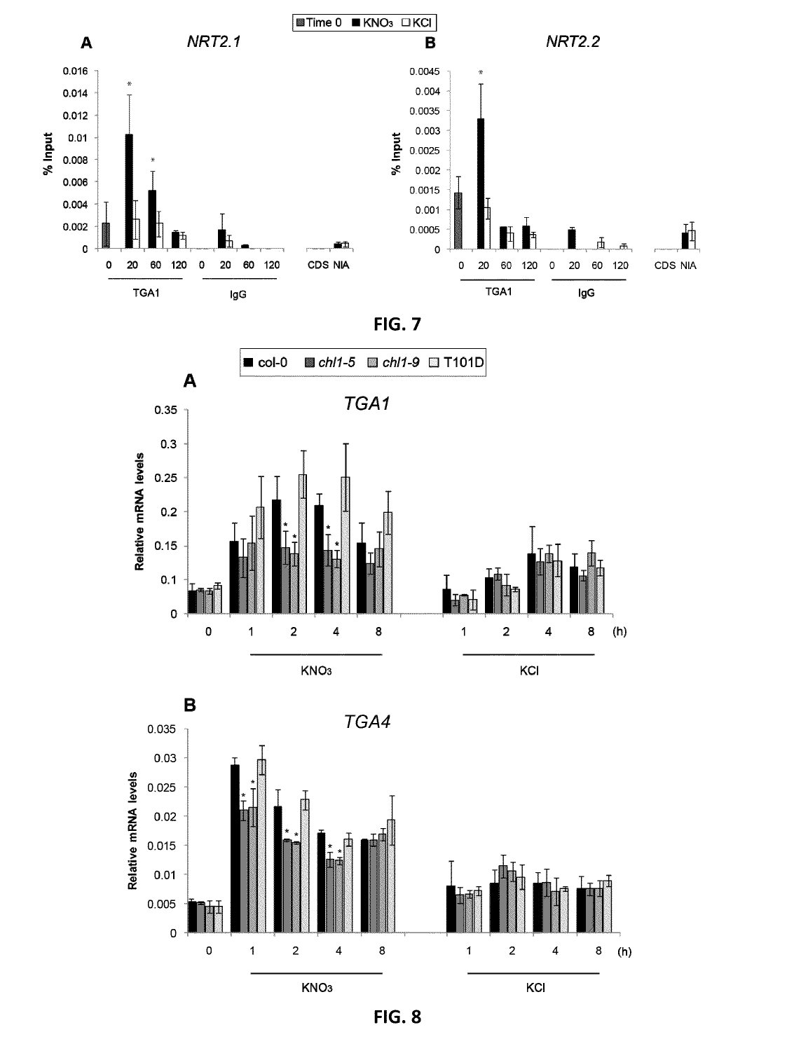

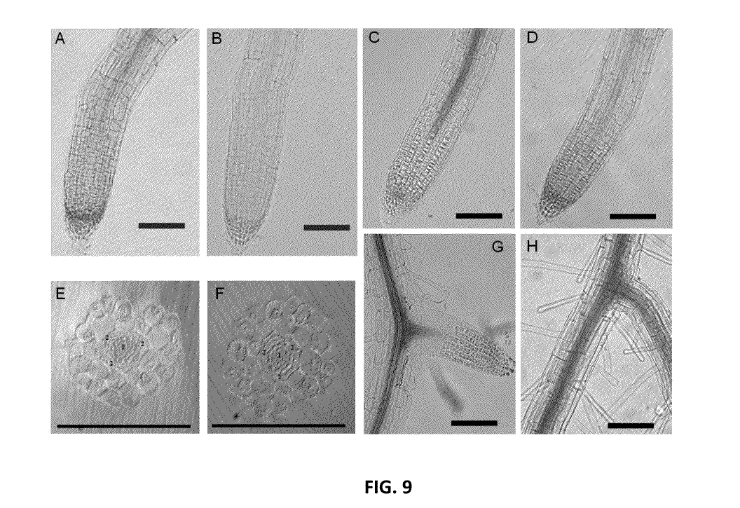

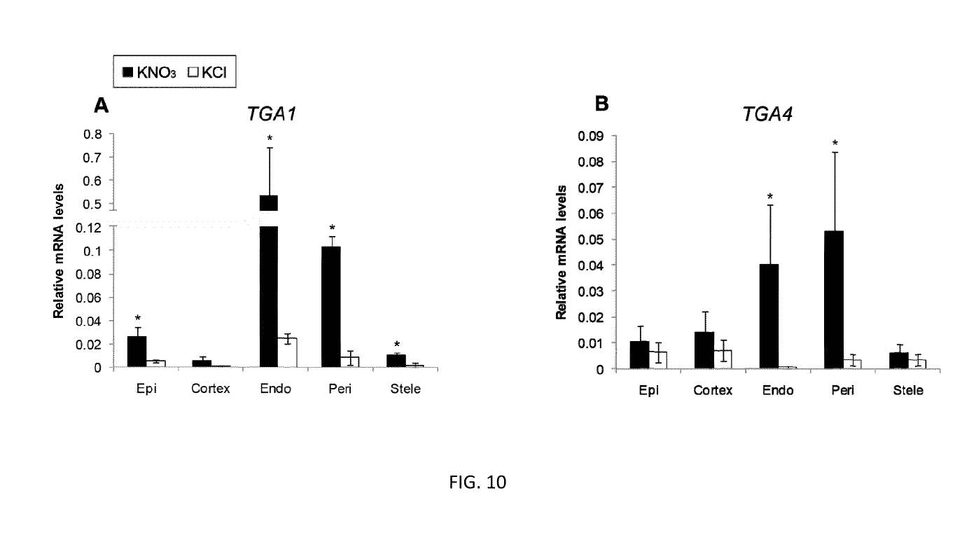

In wild-type plants, all four genes, NRT2.1, NRT2.2, NIA1 and NIR, were highly-induced by nitrate treatment. However, the nitrate induction of NRT2.1, NRT2.2, and NIR genes was significantly lower in the tga1/tga4 mutant. FIG. 6A-B (25% and 48% lower 2 hrs after treatment for NRT2.1 and NRT2.2 than wild-type, respectively); FIG. 6C (41% lower 1 hr after treatment for NIR than wild-type). No difference in the expression of NIA1 was observed between wild-type and tga1/tga4 mutant plants. These results indicate that NRT2.1, NRT2.2, and NIR are regulated by TGA1 and TGA4 in response to nitrate treatment.

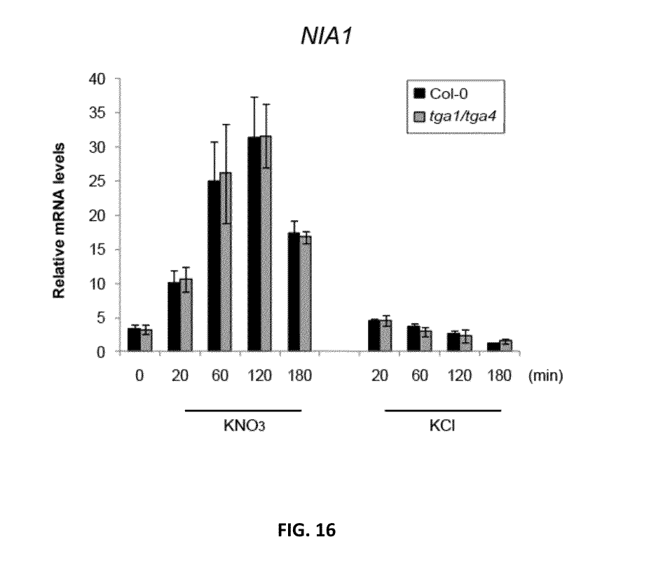

In order to determine if the expression of genes participating in nitrate reduction are regulated by TGA1 and TGA4 we evaluated the expression of NIA1 and NIR under the same experimental conditions. No difference in the expression of NIA1 was observed between wild-type and tga1/tga4 mutant plants (FIG. 16). However, the nitrate induction of NIR gene was significantly lower (41%) in the tga1/tga4 mutant 1 hour after nitrate treatment (FIG. 6C). These results indicate NRT2.1, NRT2.2 and NIR are target genes of TGA1 and TGA4.

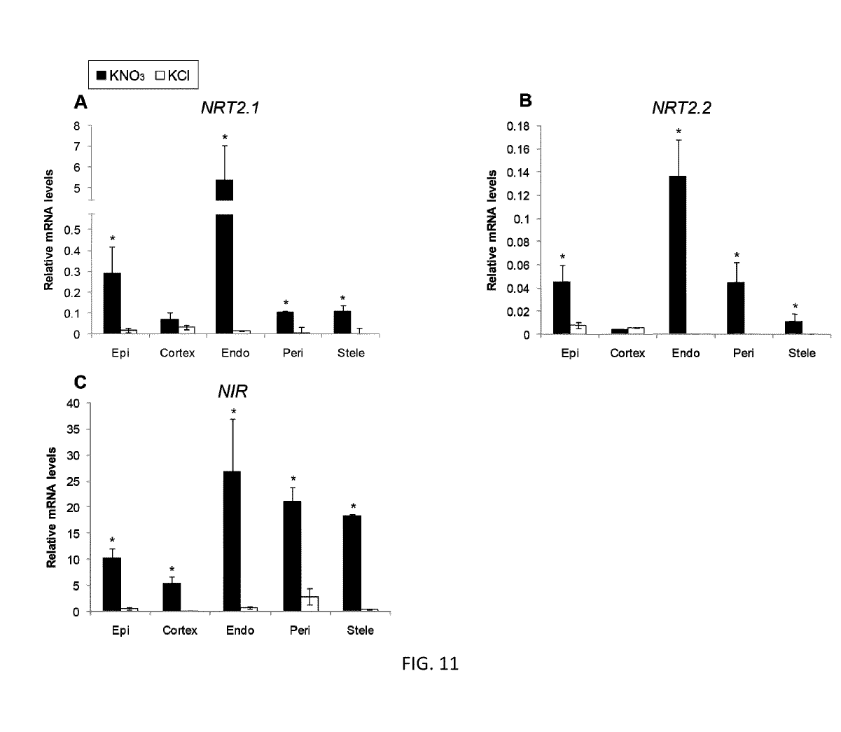

Since TGA1 and TGA4 are regulated in a tissue specific manner, we evaluated the root cell-specific expression of TGA1/TGA4 target genes in response to nitrate treatments. FIGS. 11A and 11B show that NRT2.1 and NRT2.2 are regulated by nitrate in epidermis, endodermis pericycle and stele. In contrast, NIR is regulated by nitrate in all cell types. Although there is overlap between TGA1/TGA4 expression domain and their target genes, additional regulatory factors are required to modulate the tissue-specific pattern of NRT2.1, NRT2.2 and NIR in response to nitrate.

### Example IX

### Effect of TGA1 on NRT2.1 and NRT2.2 Expression

The network model also predicted a direct effect of TGA1 on the expression of NRT2.2. A previous report showed that NRT2.1 and NRT2.2 are located very closely within the Arabidopsis genome. Orsel et al. (2002) Plant Physiol. 129:886-96. Another study proposed that a similar transcriptional mechanism is involved in the nitrate response of NRT2.1 and NRT2.2. Girin et al. (2007) Plant Cell Environ. 30:1366-80. In order to determine if NRT2.1 is a direct target of TGA1, the promoter region of NRT2.1 was manually inspected, and it was found that a TGA1 binding motif (Schindler et al. (1992) Plant Cell 4:1309-19) resides between position -309 and -304 from the transcription start site. These findings suggest that the expression of NRT2.1 and NRT2.2 is directly regulated by TGA1.

To verify that expression of NRT2.1 and NRT2.2 is directly regulated by TGA1, chromatin immunoprecipitation (ChIP) assays were performed using a TGA1-specific antibody and a non-specific IgG as a negative control. Plants were treated with 5 mM KNO₃ or 5 mM KCl for 20, 60, or 120 min at dawn of day 15. Immunoprecipitated DNA was quantified by qPCR using specific primers designed against the NRT2.1 or NRT2.2 promoter regions containing the TGA1 binding motif. TGA1 binds to the NRT2.1 and NRT2.2 promoter in a nitrate-dependent manner. FIG. 7. These specific occupancies were not observed in the immunoprecipitation with the unspecific IgG, or in the KCl-treated plant samples. Thus, TGA1 is a transcription factor that directly regulates expression of the NRT2.1 and NRT2.2 genes in response to nitrate treatments. Moreover, as soon as 20 minutes after nitrate treatments, TGA1 was detected in the promoter region of its target genes, suggesting that TGA1 and TGA4 play a role in the early response to nitrate/nitrite.

### Example X

### TGA1/TGA4 Phenotypes are Not Due to a Defect in Nitrate Uptake

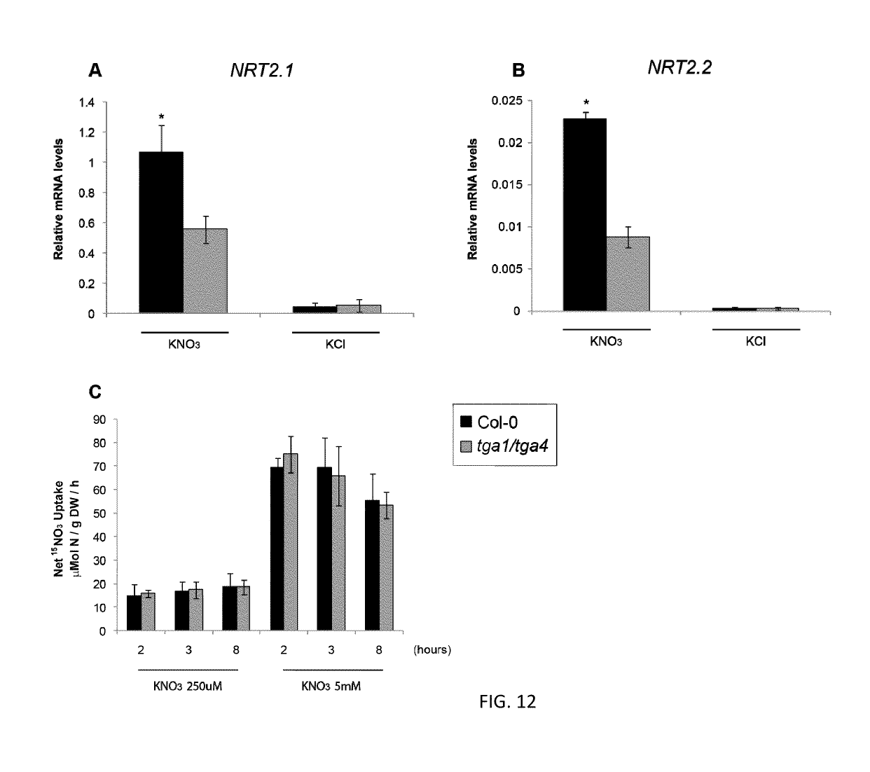

NRT2.1 and NRT2.2 are part of the high affinity transport system (HATS) which is necessary for nitrate uptake under low nitrate concentrations (Li et al., 2007). Hu et at demonstrated that NRT2.1 is induced by a wide range of nitrate concentrations and the NRT2.1 nitrate response is composed of a low and high affinity phases (Hu et al., 2009). As shown in FIGS. 6A and 6B, TGA1 and TGA4 are necessary for nitrate induction of NRT2.1 and NRT2.2 under 5 mM KNO3 treatments, a concentration in the low affinity range. In order to explore if TGA1 and TGA4 are involved in the nitrate induction of NRT2.1 and NRT2.2 in the high affinity phase, we evaluated NRT2.1 and NRT2.2 gene expression 2 hours after 250 μM KNO3 or 250 μM KCl treatments. FIGS. 12A and 12B show that TGA1 and TGA4 are necessary for nitrate induction of NRT2.1 and NRT2.2 under 250 μM KNO3 treatments. These results indicate that TGA1 and TGA4 are positive regulators of NRT2.1 and NRT2.2 gene expression in both low and high affinity phases.

To determine whether the reduced expression of NRT2.1 and NRT2.2 in the tga1/tga4 double mutant impact nitrate uptake, we performed net nitrate uptake experiments using ¹⁵NO₃⁻ isotope labeling. Plants were grown hydroponically as described above and treated with 250 μM or 5 mM NO₃⁻ enriched with 10% ¹⁵NO₃⁻ for the indicated time. Net nitrate uptake was found to be similar in wild-type and tga1/tga4 double mutant plants (FIG. 12C). Since we did not observe differences in nitrate uptake between wild-type and tga1/tga4 in a long term ¹⁵NO₃⁻ exposure (8 hours) (FIG. 12C), the observed tga1/tga4 phenotypes showed in FIGS. 3A and 3B are likely not due to a defect in nitrate absorption. This result suggests that the effect on gene expression in response to nitrate in the tga1/tga4 is likely due to a defect in a signalling pathway.

### Example XI

### TGA1 Binds to NRT2.1 and NRT2.2 Promoters in a Nitrate Dependent Manner

The network model (FIG. 5) predicts a direct effect of TGA1/TGA4 on the expression of NRT2.2. In order to determine if NRT2.1 is also a direct target of TGA1/TGA4 we manually inspected the promoter region of NRT2.1 and found two of the previously described TGA1 binding motif (Schindler et al., 1992) between position -1338 and -1333 and -371 and -266 from its translation start site. Interestingly, Girin et al., 2007 made deletions of NRT2.1 promoter to identify regions that control nitrate induction and they observed a strong decreased in gene expression in response to nitrate when the region between -456 and -245 was deleted (Girin et al., 2007). Hence, TGA1 binding site is contained in a region of NRT2.1 promoter that is important for nitrate induction of gene expression. We used chromatin immunoprecipitation (ChIP) assays using a TGA1 specific antibody and a non-specific IgG as a negative control to evaluate if NRT2.1 and NRT2.2 are direct targets genes of TGA1. Plants were treated with 5 mM KNO₃ or 5 mM KCl as negative control, for 20, 60, and 120 min at dawn of the 15^{th} day as done above.

Immunoprecipitated DNA was quantified by qPCR using specific primers designed against the NRT2.1 promoter region containing the TGA1 binding motif in positions -371 and -366 or NRT2.2 promoter regions containing two TGA1 binding motif in positions -1287 and -1282 and -1194 and -1189. As shown in FIGS. 7A and 7B, TGA1 is bound to the NRT2.1 and NRT2.2 promoters in a nitrate-dependent manner. This binding is specific for NRT2.1 and NRT2.2 promoter region and not to other regions of the genes since no amplification was observed when we used primers designed against NRT2.1 and NRT2.2 coding sequence (FIG. 7). NRT2.1 expression levels are three-fold higher than NRT2.2 (FIG. 6) in response to nitrate and accordingly TGA1 is recruited more to the NRT2.1 than NRT2.2 promoter regions. No occupancy was observed when we amplified NIA1 promoter regions, a nitrate responsive gene that is not regulated by TGA1 and TGA4 (FIG. 7). TGA1 occupancies was not observed in the immunoprecipitation with the unspecific IgG or in the control condition with KCl. This result indicates TGA1 is recruited to the promoter region of NRT2.1 and NRT2.2 upon nitrate treatments to regulate their expression.

### Example XII

### The Expression of NRT2.1 in Response to Nitrate is Affected in Chl1-5 and T101D Mutants

Col-0, chl1-5,chl1-9 and T101D plants were grown hydroponically using the experimental conditions described in Materials and Methods. At the beginning of the light period of the 15th day, plants were treated with 5 mM KNO₃ or 5 mM KCl as control, for the indicated times. RNA was isolated and NRT2.1 mRNA levels measured by RT-qPCR. The clathrin gene (At4g24550) was used as a normalization reference. Values plotted correspond to the mean of three independent biological replicates±standard deviation. (FIG. 15) The asterisk indicates means that significantly differ between mutant lines and col-0 (P<0.05).

### Example XII

### Construction of pTGA1:GUS and pTGA4:GUS Gene Fusion and GUS Activity Assays

For the chimeric pTGA1:GUS and pTGA4:GUS gene fusion, a 2000 bp fragment upstream of the TGA1 and TGA4 translational start codon was amplified from genomic DNA from the A. thaliana ecotype Col-0. The following primers were used to amplify TGA1 and TGA4 promoter and were designed to introduce a BamHI and NcoI sites: TGA1 promoter (forward,5′-TTGGATCCTTACTACGTCACCAGAATC (SEQ ID NO:21) and reverse,5′-AACCATGGTTTTCCTCAACTGAAAACAAAG (SEQ ID NO:22)) and TGA4 promoter (forward, 5′-TTGGATCCAGAAGTTGTGGTCACC (SEQ ID NO:23) and reverse, 5′-AACCATGGATTTCTTCAACTAGCAAC (SEQ ID NO:24)). Recombinant plasmids were digested with BamHI and NcoI, and DNA fragments were ligated into pCAMBIA 1381 (CAMBIA, Canberra, Australia). The structure of the constructs was verified by DNA sequencing. The constructs were then introduced into Agrobacterium tumefaciens GV3101 by electroporation. A. tumefaciens-mediated transformation of Arabidopsis plants was accomplished using the floral dip protocol (Clough and Bent, 1998). Seeds of the T1 generation were selected for resistance to hygromycin. At least 8 independent transgenic lines were obtained for each construct, and transgene presence was verified by PCR. For histochemical analysis of GUS activity, seedlings were incubated at 37° C. in a GUS reaction buffer (100 mM sodium phosphate buffer, pH 7.0, 0.5 mM potassium ferricyanide, 0.5 mM potassium ferrocyanide, 0.1% (vol/vol) Triton X-100, 0.1% (wt/vol) sodium lauroylsarcosine) plus 1 mM 5-bromo-4-chloro-3-indolyl-β-D-glucuronide (X-Gluc). After staining, samples were cleared by incubation with 0.24 N HCl in 20% methanol at 57° C. for 15 minutes. This solution was replaced with 7% NaOH, 7% hydroxylamine-HCl in 60% ethanol and incubated for 15 minutes at room temperature. Seedlings were then rehydrated for 5 minutes in each 40%, 20% and 10% ethanol, and infiltrated for 15 minutes in 5% ethanol, 25% glycerol. Samples were mounted in 50% glycerol on glass microscope slides and were imaged using DIC optics on a Nikon Eclipse 80i microscope. For each marker line and treatment, at least 15 plants were analyzed.

What may be claimed is:

1. A method for increasing nutrient efficiency in a plant, the method comprising:
introducing at least one heterologous polypeptide into root tissue of the plant, wherein said heterologous polypeptide is selected from the group consisting of TGA1, TGA4 and combinations thereof.

2. The method according to claim 1 wherein if the heterologous polypeptide is TGA 1, the heterologous polypeptide is selected from the group consisting of SEQ ID NO. 1.

3. The method according to claim 1 wherein if the heterologous polypeptide is TGA 4, the heterologous polypeptide is selected from the group consisting of SEQ ID NO. 11.

4. The method according to claim 1 wherein said increased nutrient use efficiency comprises increased efficiency in regulating nitrate as compared to a plant of the same species.

5. A method for increasing root growth in a plant, the method comprising:
introducing a heterologous TGA1 and/or a TGA4 polypeptide into root tissue of the plant, thereby producing a modified plant comprising the heterologous TGA1 and/or a TGA4 polypeptide, wherein the modified plant comprises increased root growth, as compared to a plant of the same species without the heterologous TGA1 and/or a TGA4 polypeptide.

6. The method according to claim 5, wherein the root growth is primary or lateral root growth.

7. The method according to claim 5, wherein introducing the heterologous TGA1 and/or a TGA4 polypeptide comprises introducing a nucleic acid into the root tissue, wherein the nucleic acid comprises a nucleotide sequence encoding the heterologous TGA1 and/or a TGA4 polypeptide.

8. The method according to claim 5, wherein the method comprises growing the modified plant in nitrogen limiting conditions.

9. The method according to claim 5, wherein the heterologous polypeptide is a TGA1 transcription factor.

10. The method according to claim 9, wherein the TGA1 transcription factor comprises an amino acid sequence that is at least 90% identical to SEQ ID NO:1.

11. The method according to claim 5, wherein the heterologous polypeptide is a TGA4 transcription factor.

12. The method according to claim 11, wherein the TGA4 transcription factor comprises an amino acid sequence that is at least 90% identical to SEQ ID NO:11.

13. The method according to claim 7, wherein the TGA1 transcription factor nucleotide sequence comprises a nucleotide sequence that is at least 90% identical to SEQ ID NO:15 or a nucleic acid sequence that hybridizes to a nucleic acid consisting of SEQ ID NO:15.

14. The method according to claim 7, wherein the TGA4 transcription factor nucleotide sequence comprises a nucleotide sequence that is at least 90% identical to SEQ ID NO:16 or a nucleic acid sequence that hybridizes to a nucleic acid consisting of SEQ ID NO:16.

15. The method according to claim 1, wherein the root tissue is a root cell.

16. The method according to claim 7, wherein the nucleotide sequence encoding the heterologous TGA1 and/or a TGA4 polypeptide is operably linked to a root tissue-specific promoter.

17. A modified plant produced by the method according to claim 1.

18. Seed produced from the modified plant of claim 17.

19. A nucleic acid molecule comprising a nucleic acid sequence having an agronomic function, wherein the nucleic acid sequence is selected from the group consisting of a nucleotide sequence that is at least 90% identical to SEQ ID NO:15., a nucleic acid sequence that hybridizes to a nucleic acid consisting of SEQ ID NO:15, a nucleotide sequence that is at least 90% identical to SEQ ID NO:16, and a nucleic acid sequence that hybridizes to a nucleic acid consisting of SEQ ID NO:16, wherein said nucleic acid sequence is operably linked to a heterologous transcribable polynucleotide molecule.

20. A transgenic plant cell stably transformed with the nucleic acid molecule of claim 19.

21. A method for producing a transgenic plant, the method comprising:
introducing a nucleic acid into root tissue of the plant, wherein the nucleic acid comprises a nucleotide sequence encoding a heterologous TGA1 and/or a TGA4 polypeptide, thereby producing a transgenic plant.

22. A transgenic plant produced by the method according to claim 21.

23. Seed produced from the transgenic plant of claim 22.

24. The transgenic plant of claim 22, wherein the plant comprises increased expression of a gene set forth in FIG. 5, as compared to a wild-type plant of the same species.

25. The transgenic plant of claim 22, wherein the gene is nitrate transporter NRT2.1, nitrate transporter NRT2.2, or nitrite reductase (NIR).

26. The transgenic plant of claim 22, wherein the transgenic plant comprises increased primary and/or lateral root growth, as compared to a wild-type plant of the same species.


SEQUENCE LISTING (24 sequences)

SEQ ID NO: 1 - protein, 368 aa, Arabidopsis thaliana
MNSTSTHFVPPRRVGIYEPVHQFGMWGESFKSNISNGTMNTPNHIIIPNNQKLDNNVSEDTSHGTAGTPHMFDQEASTSRHPDKIQRRLAQNREAARKSRLRKKAYVQQLETSRLKLIQLEQELDRARQQGFYVGNGIDTNSLGFSETMNPGIAAFEMEYGHWVEEQNRQICELRTVLHGHINDIELRSLVENAMKHYFELFRMKSSAAKADVFFVMSGMWRTSAERFFLWIGGFRPSDLLKVLLPHFDVLTDQQLLDVCNLKQSCQQAEDALTQGMEKLQHTLADCVAAGQLGEGSYIPQVNSAMDRLEALVSFVNQADHLRHETLQQMYRILTTRQAARGLLALGEYFQRLRALSSSWATRHREPT

SEQ ID NO: 2 - protein, 365 aa, Thellungiella halophila
MNTTSTHFVTPRRFEIYEPLNQIGMWEESFKNNGGMYTPNSIIIPTNEKPDSLSEDTSHGTEGTTPHKFDQEASTSRHPDKVQRRLAQNREAARKSRLRKKAYVQQLETSRLKLIQLEQELDRARQQGFYVGNGVDTNALGFSDNISSGIVAFEMEYGHWVEEQNRQISELRTVLHGQVSDVELRSLVETAMKHYVQLFRMKSAAAKIDVFYIMSGMWKTSAERFFLWIGGFRPSELLKVLLPHFDPLTDQQVLNVCNLRKSCQQAEDAVSQGMEKLQHTLTESVAAGKLGEGSYIPQITCAMERLEALVSFVNHADHLRHETLQQMHRILTTRQAARGLLALGEYFQRLRALSSSWATRQREPT

SEQ ID NO: 3 - protein, 364 aa, Arabidopsis lyrata
MNTTSTHFVPPRRFEVYEPLNQIGMWEESFKNNGGMYTPGSIIIPTNEKPDSLSEDTSHGTEGTPHKFDQEASTSRHPDKIQRRLAQNREAARKSRLRKKAYVQQLETSRLKLIHLEQELDHARQQGFYVGNGVDSNALCFSDNMSSGIVAFEMEYGHWVEEQNRQISELRTVLHGQVSDIELRSLVENAMKHYFQLFRMKSAAAKIDVFYVMSGMWKTSAERFFLWIGGFRPSELLKVLLPHFDPLTDQQLLDVCNLRQSCQQAEDALSQGMEKLQHTLAESVAAGKLCEGSYIPQMTCAMERLEALVSFVNQADHLRHETLQQMHRILTTRQAARGLLALGEYFQRLRALSSSWAARQREPT

SEQ ID NO: 4 - protein, 364 aa, Brassica rapa
MNTTTSTHFVPPTRFEIYDPLNQIGTMWEESFKNNGGGFYTPNSIIIPTNQKPYSLSEDGTEGTPHKFDQEASTSRHPDKTQRRLAQNREAAKKSRLRKKAYVQQLETSRLKLIHLEQELDRARQQGFYASNRVDTNALSFSDNMCSGIVAFEMEYGHWVEEQNRQISELRTVLNGQVSDIELRLLVDNAMKHYFQLFRMKSAAAKLDVFYIMSGMWKTSAERFFLWIGGFRPSELLKVLLPHFDPMMDQQVLDVCNLRQSCQQAEDAVSQGMEKLQHTLAESVAAGELGEGSYVPQITSAMERLEALVSFVNQADHLRHETLQQMHRILTTRQAARGLLALGEYFQRLRALSSSWETRQREPT

SEQ ID NO: 5 - protein, 390 aa, Arabidopsis arenosa
MNTTSTHFVPPRRFEVYEPLNQIGMWEESFKNNGGMYTPGSIIIPTNEKPDSLKLMRSLIFVQSEDTSHGTEGTPHKFDQEASTSRHPDKIQRRLAQNREAARKSRLRKKAYVQQLETSRLKLIHLEQELDHARQQGFYVGNGVDSNALGFSDNMSSGIVAFEMEYGHWVEEQNRQISELRTVLHGQVSDIELRSLVENAMKHYFQLFRMKSAAAKIDVFYVMSGMWKTSAERFFLWIGGFRPSELLKVLLPHFDPLTDQQLLDVCNLRQSCQQAEDALSQGMEKLQHTLAESVAAGKLGEGSYIPQMTCAMERLEALVSFVNQADHLRHETLQQMHRILTTRQAARAEDALSQGMEKLQQYISRECSSWETWRRKLYSSNDLCYGEIGG

SEQ ID NO: 6 - protein, 362 aa, Vitis vinifera
MNSSSTHFVTSRRMGIYEPLHQISTWGESFKTNGCPNTSASTIAELEAKLDNQSEDTSHGTPGPSDKYDQEATKPVDKVQRRLAQNREAARKSRLRKKAYVQELESSRVKLMQLEQELERARQQGLYIGGGLDAGHLGFSGAVNSGIAAFEMEYGHWVEEQSSQICELRTALHAHISDVELRILVETAMNHYFNLFRMKANAAKADVFYMMSGMWKTSAERFFLWIGGFRPSELLKVLVPQLDPLTDQQILDVCNLRQSCQQAEDALTQGMEKLQQILAEAVAAGQLGEGSYIPQLATALEKLEAVVSFVNQADHLRQETLQQMVRILTVRQAARGLLALGEYFQRLRALSSLWATRPREPA

SEQ ID NO: 7 - protein, 362 aa, Phaseolus vulgaris
MNSASPQFVSARRMSVYDPIHQISMWGEGFKSNGNLSASMPLIDDADMKLDSQSEDASHGILGAPSKYDQEANKPTDKIQRRLAQNREAARKSRLRKKAYVQQLESSRLKLMQLEQELERARHQGMYIGGGLDSNHMGFSGSVNSGITTFEMEYGHWVNEQNRQITELRTALNAHIGDIELRILVDGMMNHYAEIFRMKSAAAKADVFYVMSGMWKTTAERFFLWIGGFRPSELLKVLGPLIEPLTEKQRLDIYNLGQSCQQAEDALSQGMDKLRHTLADSVAAGQFMEGTYIPQMTSAMEKLEALVSFVNQADHLRQGTLQQMSRILTIRQAARCLLALGEYFQRLRALSSLWSNRPREPA

SEQ ID NO: 8 - protein, 363 aa, Medicago truncatula
MNSPSAQFVSSRRMSVYDPIHQINMWGEGFKSNGNLSASIPLIDEADLKFDSSQSEDASHGMLGTSNKYEQEANRPIDKIQRRLAQNREAARKSRLRKKAYVQQLESSRLKLVQLEQELERVRQQGMYMGGGLDSNNMCFAGPVNPGIAAFEMEYGHWVDEQNRQISEMRNALNSHISDIELRMLVDGMMNHYAEIYRMKSAAAKTDVFYVMSGMWKTTAERFFLWIGGFRPSELLKILGPMIEPLTEQQRLDIDNLGQSCQQAEDALSQGMEKLRQTLADSVAAGQFIEGTYIPQMATAMEKLEALVSFVNQADHLRQETLQQMSRTLTIRQSARCLLALGEYFQRLRALSSLWSNRPREPA

SEQ ID NO: 9 - protein, 374 aa, Glycine max
MDATSSQFVSSRRMGVYDPIHQISMWEETFKSNDTNNLTVSTSIIGEVEMKLDNQVHVQSEDASHGIFGTSVKYDQDANRLTDKTQRRLAQNREAARKSRLRKKAYVQQLESCRLKLVQLEQEVDHAKQQGLYIGDGLGSNNLGFAGSVNSGITLFKMEYGNWLEEQNRQILELRTALSSHIGDIQLGTLVQGIMNHYTKLFSMKSAAAKADVFYVMSGMWKTTAERFFLWIGGFRPSELLKVLVPLSEPLTEQQRFDAYGLEKSCQQAEDALSQGMEKLQQMLADSVGPGQLVEGTHIPQMDTAMERLEALVSFVNQADHLRQETLRQMYRILTTRQTGRFLLDLGEYFQRLRALSKLWANRPQELTKSVIKH

SEQ ID NO: 10 - protein, 354 aa, Ricinus communis
MNSPSTQFVSSGRTGIYEPIHQIGMWGEPFKSNGIPNASTSMFVAGDPNSSQSIIIAVDTKLDNQSEDTSQNTLGPSSKYDQEATKPIDKVQRRLAQNREAARKSRLQKKAYVQQLESSRLKLIQIEQELERARQQGLNIGGGVETSHLGFAGPNNSGIATFEMEYGHWLEEQNRQIGDLRTALNAHISDIELCILVESGINHYSELFRMKATAAKADVFYLMSGMWKSSAERFFLWIGGFRPSELLKILKPQLEPLTDQQLLDVCNLKQSCQQAEDALSQGMEKLQQTLVEAVAAGRLGEASHLPQMDTAMEKLEGLVRFVQQKDLVSSLLECIFLPLSISADINFLCPSLSL

SEQ ID NO: 11 - protein, 364 aa, Arabidopsis thaliana
MNTTSTHFVPPRRFEVYEPLNQIGMWEESFKNNGDMYTPGSIIIPTNEKPDSLSEDTSHGTEGTPHKFDQEASTSRHPDKIQRRLAQNREAARKSRLRKKAYVQQLETSRLKLIHLEQELDRARQQGFYVGNGVDTNALSFSDNMSSGIVAFEMEYGHWVEEQNRQICELRTVLHGQVSDIELRSLVENAMKHYFQLFRMKSAAAKIDVFYVMSGMWKTSAERFFLWIGGFRPSELLKVLLPHFDPLTDQQLLDVCNLRQSCQQAEDALSQGMEKLQHTLAESVAAGKLGEGSYIPQMTCAMERLEALVSFVNQADHLRHETLQQMHRILTTRQAARGLLALGEYFQRLRALSSSWAARQREPT

SEQ ID NO: 12 - protein, 332 aa, Medicago truncatula
MGRTFKSNGDSSVYEPEMKLNNQSEDASFGILGTSIKYDHQEANKVTNKMQRRLAQNREAARKSRLKKKAHIQQLESCRLKLLQVEQELDHTKQGLYIGGGLDSNNLGFAGSVNSEIATFKMEYEHWVEELNRQMLELKGALSAHSSDIRIGELVNGLMNHYFKLFCMKSDAAKVDVFYVITGMWKTTAEGFFLWIGGFRPSELLKVLVPLIEPLTEEQRFDAYNLEKSCRQAEDALSQGMEKLQGMLVDTVAAGQLVEGTYIPQMDIAIERLEALASFVNQADHLRQETLQQMSRILTVRQTARWLLALGEYFQRLRDLSKLWTNRPREPA

SEQ ID NO: 13 - protein, 361 aa, Vitis vinifera
MSSSSTQFATSRRIGMHEPLHQISMWRDTFKGDSNPITGASTIMQVDTMLDNKSESTSHDSLGPSGNSQPEDRTTDKTQRRLAQNREAARKSRLRKKAYVQQLETSRLKLTELEQELERARQQGLYIGGSLDTTRVGFSGTINSGIATFEMEYGHWVEEQHRQNCELRNALQAHVTDIELRILVESALNHYYELFRMKADAAKADVFYLMSGMWRTSAERFFLWIGGFRPSELLNVLMPHFEPLTDQQLLDVCNLRQSSQQAEDALSQGMDKLQQTLAQSIVTDPVGAGNYRSQMAEAVEKLDALESFVNQADHLRQQTLRQMSHLLTTRQAARGLLALGEYFHRLRALSSLWAARPREPA

SEQ ID NO: 14 - protein, 371 aa, Zea mays
MMMSSSSSNTQAVPFRDMGMYEPFQQLSGWENTFNTITTNNHNNNNQTSSTIARTEADANNKGNYTCLYNNSVEAEPSGNNDQGEVQISDKMKRRLAQNREAARKSRLRKKAHVQQLEESRLKLSQLEQELVRARQQGLCVVTSDATYLGPAGTMNTGIAAFEMEHKHWLEEQSKRVSEIRTALQAHISDVELKMLVDVCLNHYANLFRMKAAAAKADVFFLISGMWRTSTERFFQWIGGFRPSELLNVVMPYIEPLTDQQLLEVTNLQQSSQQAEEALSQGLDKLQQGLVENIAVVESLNHGGAQMASAMENLESLEGFVNQADHLRKQSLQQMSKVLTTRQAARGLLALGEYFHRLRALSSLWAARPRN

SEQ ID NO: 15 - DNA, 1107 bp, Arabidopsis thaliana
atgaattcgacatcgacacattttgtgccaccgagaagagttggtatatacgaacctgtccatcaattcggtatgtggggggagagtttcaaaagcaatattagcaatgggactatgaacacaccaaaccacataataataccgaataatcagaaactagacaacaacgtgtcagaggatacttcccatggaacagcaggaactcctcacatgttcgatcaagaagcttcaacgtctagacatcccgataagatacaaagacggcttgctcaaaaccgcgaggctgctaggaaaagtcgcttgcgcaagaaggcttatgttcagcaactggaaacaagcaggttgaagctaattcaattagagcaagaactcgatcgtgctagacaacagggattctatgtaggaaacggaatagatactaattctctcggtttttcggaaaccatgaatccagggattgctgcatttgaaatggaatatggacattgggttgaagaacagaacagacagatatgtgaactaagaacagttttacacggacacattaacgatatcgagcttcgttcgctagtcgaaaacgccatgaaacattactttgagcttttccggatgaaatcgtctgctgccaaagccgatgtcttcttcgtcatgtcagggatgtggagaacttcagcagaacgattcttcttatggattggcggatttcgaccctccgatcttctcaaggttcttttgccacattttgatgtcttgacggatcaacaacttctagatgtatgcaatctaaaacaatcgtgtcagcaagcagaagacgcgttgactcaaggtatggagaagctgcaacacacccttgcggactgcgttgcagcgggacaactcggtgaaggaagttacattcctcaggtgaattctgctatggatagattagaagctttggtcagtttcgtaaatcaggctgatcacttgagacatgaaacattgcaacaaatgtatcggatattgacaacgcgacaagcggctcgaggattattagctcttggtgagtattttcaacggcttagagccttgagctcaagttgggcaactcgacatcgtgaaccaacgtag

SEQ ID NO: 16 - DNA, 1092 bp, Arabidopsis thaliana
atgaatacaacctcgacacattttgttccaccgagaaggtttgaagtttacgagcctctcaaccaaatcggtatgtgggaagaaagtttcaagaacaatggagacatgtatacgcctggctctatcataatcccgactaacgaaaaaccagacagcttgtcagaggatacttctcatgggacagaaggaactcctcacaagtttgaccaagaggcttccacatctagacatcctgataagatacagagaaggctagcacagaatcgagaggcagctaggaaaagtcgtttgcgcaagaaagcttatgttcagcagctagagactagccggttaaagctaattcatttagagcaagaactcgatcgtgctagacaacagggtttctatgtggggaacggagtagataccaatgctcttagtttctcagataacatgagctcagggattgttgcatttgagatggaatatggacattgggtggaagaacagaacaggcaaatatgtgaactaagaacggttttacatggacaagttagtgatatagagcttcgttctctagtcgagaatgccatgaaacattactttcaactcttccgaatgaagtcagccgctgcaaaaatcgatgttttctatgtcatgtccggaatgtggaaaacttcagcagagcggtttttcttgtggataggcggatttagaccctcagagcttctcaaggttctgttaccgcattttgatcctttgacggatcaacaacttttggatgtatgtaatctgaggcaatcatgtcaacaagcagaagatgcgttatcccaaggtatggagaaactgcaacatacattagcagagagtgtagcagccgggaaacttggtgaaggaagttatattcctcaaatgacttgtgctatggagagattggaggctttggtcagctttgtaaatcaagctgatcatctgagacatgagacattgcaacagatgcatcggatcttaaccacgcgacaagcggctagaggtttgttagcattaggggagtatttccaaaggcttcgagctttgagttcgagttgggcggctaggcaacgtgaaccaacg

SEQ ID NO: 17 - DNA, 27 bp, Artificial
  AtNRT2.1 forward primer
ctatcctgtatcactgtatgtaaccag

SEQ ID NO: 18 - DNA, 27 bp, Artificial
  AtNRT2.1 reverse primer
ggatggatagtcaacaatatggttgtg

SEQ ID NO: 19 - DNA, 20 bp, Artificial
  AtNRT2.2 forward primer
ctcaacagagggaacaccgg

SEQ ID NO: 20 - DNA, 25 bp, Artificial
  AtNRT2.2 reverse primer
cccaaaatatattacaatgtagttg

SEQ ID NO: 21 - DNA, 27 bp, Artificial
  TGA1 forward primer
ttggatccttactacgtcaccagaatc

SEQ ID NO: 22 - DNA, 30 bp, Artificial
  TGA1 reverse primer
aaccatggttttcctcaactgaaaacaaag

SEQ ID NO: 23 - DNA, 24 bp, Artificial
  TGA4 forward primer
ttggatccagaagttgtggtcacc

SEQ ID NO: 24 - DNA, 26 bp, Artificial
  TGA4 reverse primer
aaccatggatttcttcaactagcaac